(19) United States
(12) Patent Application Publication
(10) Pub. No.: US 2022/0000729 A1
(43) Pub. Date: Jan. 6, 2022

(54) METHOD FOR TREATING HAIR, COMPRISING THE APPLICATION OF COATED PIGMENTS AND SILANES

(71) Applicant: Henkel AG & Co. KGaA, Duesseldorf (DE)

(72) Inventors: MARC NOWOTTNY, Moenchengladbach (DE); JUERGEN SCHOEPGENS, Schwalmtal (DE); TORSTEN LECHNER, Langenfeld (DE)

(73) Assignee: Henkel AG & Co. KGaA, Duesseldorf (DE)

(21) Appl. No.: 17/295,284

(22) PCT Filed: Aug. 26, 2019
(86) PCT No.: PCT/EP2019/072690

§ 371 Date: May 19, 2021

(51) Int. Cl. A61K 8/11; A61Q 5/06; A61K 8/58; A61K 8/41

(52) U.S. Cl. CPC A61K 8/11; A61Q 5/065; A61K 8/585; A61K 2800/622; A61K 2800/43; A61K 2800/88; A61K 8/418

(57) ABSTRACT

A process for dyeing keratinous material, in particular human hair, comprising
(a) applying an agent containing a pigment with a colored core and a silicon-containing coating, and
(b) simultaneously or successively applying an agent containing an organic silicon compound with one, two or three silicon atoms.

### CROSS-REFERENCE TO RELATED APPLICATION

This application is a U.S. National-Stage entry under 35 U.S.C. § 371 based on International Application No. PCT/EP2019/072690, filed Aug. 26, 2019, which was published under PCT Article 21(2) and which claims priority to German Application No. 10 2018 219 876.4, filed Nov. 20, 2018, which are all hereby incorporated in their entirety by reference.

### TECHNICAL FIELD

The disclosure relates to a method of treating hair involving the use of coated pigments and silanes.

### BACKGROUND

The change in shape and color of keratin fibers, especially hair, is an important area of modern cosmetics. To change the hair color, the expert knows various coloring systems depending on coloring requirements. Oxidation dyes are usually used for permanent, intensive coloring with good fastness properties and good gray coverage. Such dyes usually contain oxidation dye precursors, so-called developer components and coupler components, which form the actual dyes with one another under the influence of oxidizing agents, such as hydrogen peroxide. Oxidation dyes are characterized by very long-lasting dyeing results.

When direct dyes are used, ready-made dyes diffuse from the colorant into the hair fiber. Compared to oxidative hair dyeing, the coloring obtained with direct dyes have a shorter shelf life and quicker wash ability. Dyeing with direct dyes usually remain on the hair for a period of between about 5 and about 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. Therefore, they can usually be removed again without residue by a few washes with detergents containing surfactants. Various products of this type are available on the market under the name hair mascara.

If the user wants particularly long-lasting coloring, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair.

EP 2168633 B1 deals with the task of producing long-lasting hair colorations using pigments. The text teaches that by using a combination of a pigment, an organic silicon compound, a film-forming polymer and a solvent on hair, it is possible to create colorations on hair that are particularly resistant to shampooing.

During the revision of the doctrine of EP 2168633 B1, its formulations have been adjusted. In the course it is seen that the disadvantages of these formulations consist in their not yet sufficient color intensities and insufficient wash fastness. The shampooing fastness of the colorations produced in EP 2168633 B1 accordingly always needs improvement.

### BRIEF SUMMARY

The subject of the present application is a method for the treatment of keratinous material, in particular human hair, which comprises the steps (a) and (b). Step (a) is characterized by the use of an agent on the keratinous material, in which the agent is at least one coated pigment with a colored core and a silicon-containing coating. The characteristic feature for step (b) is the use of an agent on the keratinous material, wherein the agent contains at least one organic silicon compound from the group of silanes with one, two or three silicon atoms. The agents applied in the steps (a) and (b) can be the same or different.

Another subject of this application is a product for dyeing keratinous material, especially human hair, which contains at least one coated pigment with a colored core and a silicon-containing coating and at least one silane with one, two or three silicon atoms.

A third subject of the present application is a multi-component packaging unit (kit-of-parts), comprising, in separately fabricated containers, an agent (a) containing at least one coated pigment in a colored core and a silicon-containing coating and an agent (b) containing at least one organic silicon compound from the group of silanes with one, two or three silicon atoms.

The purpose of the present disclosure was to provide a dyeing system with fastness properties comparable to those of oxidative dyeing. The color intensities and wash fastness properties in particular should be outstanding, but the use of oxidation dye precursors normally used for this purpose should be avoided. A technology was sought that would make it possible to fix the pigments known from the state of the art in an extremely durable way to the hair. Even after multiple washings (for example hair washing), the pigments placed on the keratinous material should not detach from the keratinous material.

Surprisingly, it has now turned out that the above-mentioned task can be excellently solved if keratinous materials, especially human hair, are dyed using a procedure in which at least one special coated pigment and at least one organic silane are used.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

A first object of the present disclosure is a method for coloring keratinous material, in particular human hair, comprising the following steps:
(a) use of an agent on the keratinous material, in which the agent contains at least one coated pigment with a colored core and a silicon-containing coating, and
(B) use of an agent on the keratinous material, wherein the agent contains at least one organic silicon compound from the group of silanes with one, two or three silicon atoms.

In the course of the work leading to this present disclosure, it has turned out that the—either simultaneous or successive—treatment of keratinous material with the coated pigment and the organic silicon compound enables the formation of very sturdy films on the keratin materials. Without restricting to this theory, it is assumed in this context that the use of the organic silicon compound (silane) leads to formation of a film on the keratinous material. The pigments used in the process have a silicon-containing layer or, in other words, a coating of a silicon-containing material. The silicon atoms found in this coating represent the outermost layer or the outer cover of the coated pigment, so an interaction of the silicon-containing coating with the silanes can take place. Covalent bonds or other adhesive forces can form on contact between the coated pigment and the silanes qualified for oligomerization or polymerization, as the case may be, such that the pigments can be permanently integrated into the silane film. In this manner, the keratinous material has been given extremely fast colors with good resistance to shampooing.

### Method for Dyeing Keratinous Material

Keratinous material includes hair, skin, nails (such as fingernails and/or toenails). Wool, furs and feathers also fall under the definition of keratinous material.

Preferably, keratinous material is understood to be human hair, human skin and human nails, especially fingernails and toenails. Keratinous material is understood to be human hair in particular.

The term “coloring agent” is used within the framework of this present disclosure for dyeing the keratin material, in particular the hair, caused by the use of pigments. In this coloration, the coated pigments are deposited as coloring compounds in a particularly homogeneous, even and smooth film on the surface of the keratin material. The film is formed in situ by oligomerization or polymerization of the organic silicon compound(s), wherein the coated pigments deposit in the film or aggregate with it.

Coated Pigment with a Colored Core and a Silicon-Containing Coating.

Step (a) of the method as invented is characterized by the use of an agent on the keratinous material that contains at least one coated pigment with a colored core and a silicon-containing coating.

As a result of their colored core, the coated pigments are color pigments.

Pigments used for coloring keratin material are usually particulate materials, in which the individual particles of the pigment can have a certain particle size. This particle size leads on the one hand to an even distribution of the pigments in or on the formed silane film and, on the other hand, avoids a rough hair or skin feel after application of the cosmetic product. It is therefore advantageous if the at least one pigment has an average particle size D50 of 1.0 to 50 μm, preferably 5.0 to 45 μm, preferably 10 to 40 μm, in particular 14 to 30 μm. The mean particle size D50, for example, can be determined using dynamic light scattering (DLS).

In the context of another embodiment, there is a preferred process for dyeing keratinous material, in particular human hair, comprising the following steps:
(a) use of an agent on the keratinous material, in which the agent contains at least one particulate material, the particles of which possess a colored core and a silicon-containing coating, and
(b) use of an agent on the keratinous material, wherein the agent contains at least one organic silicon compound from the group of silanes with one, two or three silicon atoms.

Within the framework of another embodiment, a particularly preferred method for dyeing keratinous material, in particular human hair, comprising the following steps:
(a) Use of an agent on the keratinous material, where the agent contains at least one particulate material, whose particles feature a mean grain size D50 from about 1.0 to about 50 μm, preferably from about 5.0 to about 45 μm, preferably from about 10 to about 40 μm, especially from about 14 to about 30 μm and possess a colored core and a silicon-containing coating, and
(b) use of an agent on the keratinous material, wherein the agent contains at least one organic silicon compound from the group of silanes with one, two or three silicon atoms.

The pigments, or the particles of the particulate material, which in their entirety form the pigment, possess a colored core. So, the inner part of the pigment or of each particle is colored. In this case, the core in the inside of the pigment/particle can be of organic or inorganic material. In a further preferred embodiment, a method as invented is characterized in that the coated pigment possesses a core of an organic or an inorganic material.

In other words, in a further preferred embodiment, a method as invented is characterized in that the particles of the particulate material possesses a core of an organic or an inorganic material.

In a particularly preferred embodiment, a method as invented is characterized in that the coated pigment possesses a core of an organic material.

Pigments within the meaning of the present disclosure are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A stir-fish is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the possibly finely dispersed pigment, the mixture is filtered. If a proportion of undissolved pigments remains on the filter paper, the solubility of the pigment is below 0.5 g/L.

It was possible to achieve particularly good results, if an organic dye pigment was inserted in the core of the coated pigment.

Within the framework of this embodiment, commercially available organic color pigments can be used, which may first be ground to reduce their particle size and then subjected to a surface treatment. In the course of this surface treatment, the silicon-containing coating is then applied on the core. The pigment obtained after the surface treatment then possesses the previously described especially low solubility in water.

The particularly well-suited organic pigments that form the core of the coated pigment can be called, for example, insoluble, organic dyes or colored lacquers, which can be selected from the group of nitroso-, intro-azo-, xanthene, anthraquinone-, isoindolinon-, isoindolin-, quinacridon-, perinon-, perylene-, diketo-pyrrolpyorrol-, indigo-, thioindido-, dioxazine-, and/or triarylmethane compounds .

Examples of particularly suitable organic pigments are carmine, quinacridone, phthalocyanine, sorghum, blue pigments with the color index numbers CI 42090, CI 69800, CI 69825, CI 73000, CI 74100, CI 74160, yellow pigments with the color index numbers CI 11680, CI 11710, CI 15985, CI 19140, CI 20040, CI 21100, CI 21108, CI 47000, CI 47005, green pigments with the color index numbers CI 61565, CI 61570, CI 74260, orange pigments with the color index numbers CI 11725, CI 15510, CI 45370, CI 71105, red pigments with the color index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In a further particularly preferred embodiment, a process as contemplated herein is characterized in that the coated pigment possesses a core of an organic material, which is selected from the group of carmine, quinacridone, phthalocyanine, sorghum, blue pigments with the color index numbers CI 42090, CI 69800, CI 69825, CI 73000, CI 74100, CI 74160, yellow pigments with the color index numbers CI 11680, CI 11710, CI 15985, CI 19140, CI 20040, CI 21100, CI 21108, CI 47000, CI 47005, green pigments with the color index numbers CI 61565, CI 61570, CI 74260, orange pigments with the color index numbers CI 11725, CI 15510, CI 45370, CI 71105, red pigments with the color index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In a further particularly preferred embodiment, a process as contemplated herein is characterized in that the particles of the particulate materials possess a core of an organic material, which is selected from the group of carmine, quinacridone, phthalocyanine, sorghum, blue pigments with the color index numbers CI 42090, CI 69800, CI 69825, CI 73000, CI 74100, CI 74160, yellow pigments with the color index numbers CI 11680, CI 11710, CI 15985, CI 19140, CI 20040, CI 21100, CI 21108, CI 47000, CI 47005, green pigments with the color index numbers CI 61565, CI 61570, CI 74260, orange pigments with the color index numbers CI 11725, CI 15510, CI 45370, CI 71105, red pigments with the color index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

Furthermore, an inorganic color pigment can be inserted in the core of the coated pigments. Within the framework of this further embodiment, commercially available inorganic color pigments can be used, which may eventually be first ground to reduce their particle size and then subjected to a surface treatment. In the course of this surface treatment, the silicon-containing coating is then applied on the core. The pigment obtained after the surface treatment then possesses the previously described especially low solubility in water.

Suitable color pigments are selected from synthetic or natural inorganic pigments. Inorganic color pigments of natural origin can be produced, for example, from chalk, ocher, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are for example colored metal oxides, hydroxides and oxide hydrates, mixed-phase pigments, sulfur-containing silicates, silicates, metal sulfides, complex metal cyanides, metal sulphates, chromates and/or molybdates. In particular, preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

Further suitable color pigments are colored pearlescent pigments. These are usually on mica- and/or micaceous base and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In another embodiment, a method as invented is characterized in that the coated pigment possesses a core of an inorganic material, which is selected from the group the colored metal oxides, metal hydroxides, metal oxihydrates, silicates, metal sulfides, complex metal cyanides, metal sulfates, bronze pigments and/or from colored pigments on mica or glitter base, which are coated with at least one metal oxide and/or a metal oxychloride.

In a further preferred embodiment, an method as invented is characterized in that the coated pigment possesses a core of an inorganic material, which is selected from pigments on mica or micaceous base, which is coated with one or more metal oxides from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfo silicates, CI 77007, pigment blue 29), chromium oxide hydrate (CI 77289), chromium oxide (CI 77288) and/or iron blue (ferric ferrocyanides, CI 77510).

Examples of particularly suitable color pigments are commercially available under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Particularly suitable color pigments with the trade name Colorona® are, for example:
Colorona Copper, Merck, MICA, CI 77491 (IRON OXIDES)
Colorona Passion Orange, Merck, Mica, CI 77491 (Iron Oxides), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
ColoronaBronze, Merck, MICA, CI 77491 (IRON OXIDES)
ColoronaBronze Fine, Merck, MICA, CI 77491 (IRON OXIDES)
ColoronaFine Gold MP 20, Merck, MICA, CI 77891 (TITANIUM DIOXIDE), CI 77491 (IRON OXIDES)
ColoronaSienna Fine, Merck, CI 77491 (IRON OXIDES), MICA
ColoronaSienna, Merck, MICA, CI 77491 (IRON OXIDES)
ColoronaPrecious Gold, Merck, Mica, CI 77891 (Titanium dioxide), Silica, CI 77491(Iron oxides), Tin oxide
ColoronaSun Gold Sparkle MP 29, Merck, MICA, TITANIUM DIOXIDE, IRON OXIDES, MICA, CI 77891, CI 77491 (EU)
ColoronaMica Black, Merck, CI 77499 (Iron oxides), Mica, CI 77891 (Titanium dioxide)
ColoronaBright Gold, Merck, Mica, CI 77891 (Titanium dioxide), CI 77491(Iron oxides)
ColoronaBlackstar Gold, Merck, MICA, CI 77499 (IRON OXIDES)

Other particularly preferred color pigments with the trade name Xirona® are for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.

Other suitable color pigments with the trade name Unipure® are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica

For the formation of the coating as invented, the previously described pigments are now treated with a surface treatment agent, before or after they were ground. The surface treatment agent(s) is/are silicon-containing substances, which form a layering, covering or a coating around the colored core. The cover or coating around the colored core is itself generally not colored, so the coloring of the keratin material is generally effected in the shading of the pigment, which forms the colored core.

In this case, the treatment with the silicon-containing surface treatment agent can be effected directly on the surface of the color pigment, or else the color pigment can beforehand be first coated with other substances such as polymers and then treated with the silicon-containing surface treatment agent In any case, the silicon-containing coating represents the outer or the outermost, as the case may be, which allows an interaction with the silanes on using the coated pigment on the keratin material.

Substances from the group of the tetra alkoxy silanes, alkyl trialkoxy silanes, dialkyl dialkoxy silanes and the trialkyl alkoxy silanes can, for example, be used as silicon-containing surface treatment agents

In another especially preferred embodiment, a method as invented is exemplified in that the coating of the coated pigments is obtained by employing a surface treatment with a surface treatment agent, wherein the surface treatment agent is selected from the group of the tetra alkoxy silanes, alkyl trialkoxy silanes, dialkyl dialkoxy silanes and the trialkyl alkoxy silanes.

The surface treatment agents from the above-mentioned groups are reactive compounds and represent silanes with at least one hydrolysable group.

For surface treatment, the said compounds can for example be dissolved or dispersed in water and/or an alcohol such as ethanol or isopropanol and then brought into contact with the pigment.

The surface treatment agents from the group of the tetra alkoxy silanes can alternatively also be designated as tetra alkyl orthosilicates. These are the tetra alkyl esters of orthosilicic acid. Particularly well-suited are tetra alkoxysilanes are compounds of the formula (O-I).

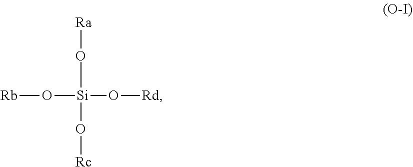

where the radicals Ra, Rb, Rc and Rd mutually independently represent linear or branched, saturated or unsaturated C₁-C₁₂-alkyl group.

The methyl group, the ethyl group, the n-propyl group, the n-butyl group, the n-pentyl group, the n-hexyl group, the n-octyl group, the n-decyl group and the n-dodecyl group can be mentioned as examples for a linear, saturated C₁-C₁₂-alkyl group. Quite specifically preferred are the methyl group and the ethyl group.

From a chain length of 3 C atoms, double-bonded alkylene groups can also be branched. The isopropyl group, the 1-(methyl) propyl group and the 1-(ethyl) propyl group can be recognized as examples for a branched, saturated C₁-C₁₂-alkyl group.

In a further specially preferred embodiment, a method as invented is exemplified in that the surface treatment agent is selected from the group of the tetra alkoxy silanes of the formula (O-I),

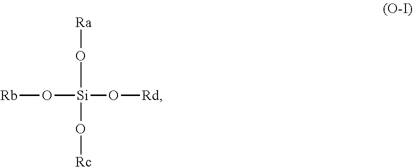

where
Ra, Rb, Rc and Rd mutually independently represent a linear or branched, saturated or unsaturated C₁-C₁₂-alkyl group, preferably an ethyl group or a methyl group.

A specifically preferred surface treatment agent from the group of tetra alkoxy silanes is the tetra ethoxy silane, which is alternatively also designated tetra ethyl orthosilicate or as silicic acid tetra ethyl ester. Tetra-ethoxy-silane has the chemical formula Si(OEt)₄ and has the CAS number 78-10-4. Tetra ethoxy silane is commercially available from the chemicals suppliers Sigma-Aldrich, VWR or Merck.

Another particularly preferred surface treatment agent from the group of tetra alkoxy silanes is the tetra methoxy silane, which is alternatively also designated tetra methyl orthosilicate or as silicic acid tetra ethyl ester. Tetra-methoxy-silane has the chemical formula Si(OMe)₄ and has the CAS number 681-84-5. Tetramethoxy silane can also be commercially sourced from chemical suppliers such as Sigma-Aldrich, VWR or Merck.

Surface treatment agents from the group of alkyl trialkoxy silanes can be selected, for example, from the group of C₁-C₁₂-alkyl-tri(C₁-C₁₂-alkoxy) silanes. Especially well-suited alkyl-trialkoxy-silanes can be selected, for example, from compounds of the formula (O-II),

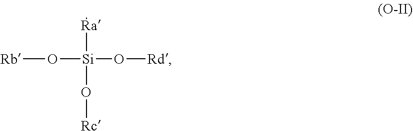

where
Ra′, Rc′ and Rd′ mutually independently represent a linear or branched, saturated or unsaturated C₁-C₁₂-alkyl group, preferably an ethyl group or a methyl group.

Examples of particularly well-suited alkyl-trialkoxy-silanes are

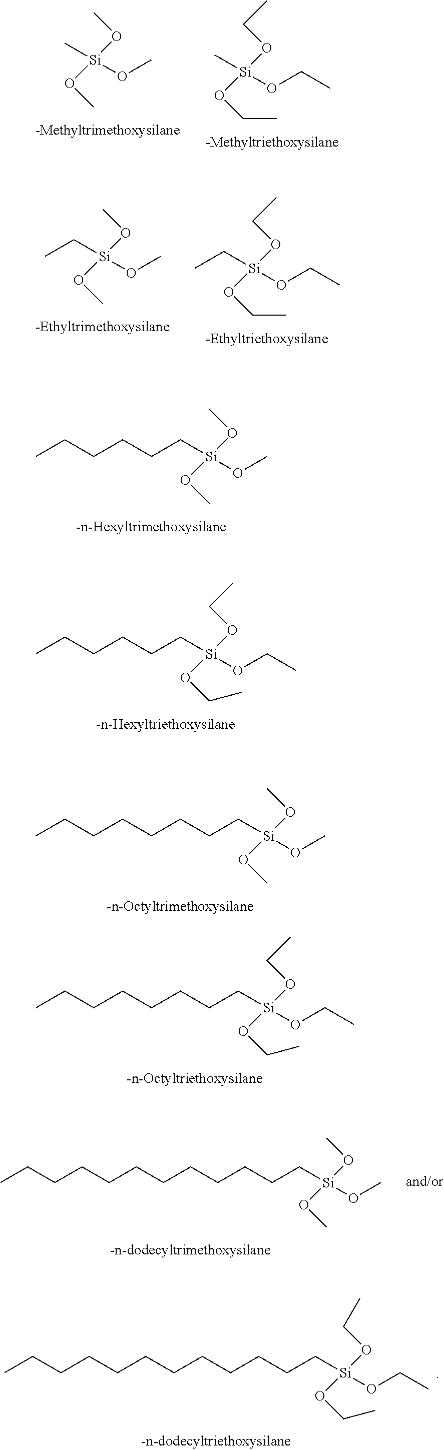

The surface treatment agents from the group of dialkyl trialkoxy silanes can be selected, for example, from the group of Di(C₁-C₁₂-alkyl)-di(C₁-C₁₂-alkoxy) silanes. Especially well-suited dialkyl-dialkoxy-silanes can be selected, for example, from compounds of the formula (O-III),

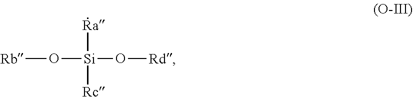

where
Ra″, Rb″, Rc″ and Rd″ mutually independently represent a linear or branched, saturated or unsaturated C₁-C₁₂-alkyl group, preferably an ethyl group or a methyl group.

A particularly well-suited dialkyl-dialkoxy silane is, for example, dimethyl diethoxy silane, alternatively also designated as diethoxy dimethyl silane, with the chemical formula (Me)₂Si(OEt)₂ and the CAS number 78-62-6. Dimethyl diethoxy silane can be commercially sourced from Sigma-Aldrich.

Surface treatment agent from the group of the trialkyl alkoxy silanes can be selected, for example, from the group of Tri(C₁-C₁₂-alkyl)-(C₁-C₁₂-alkoxy)silanes. Especially well-suited dialkyl-dialkoxy-silanes can be selected, for example, from compounds of the formula (O-IV),

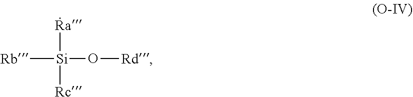

where
Ra′″, Rb′″, Rc′″ and Rd′″ mutually independently represent a linear or branched, saturated or unsaturated C₁-C₁₂-alkyl group, preferably an ethyl group or a methyl group.

A particularly well-suited trialkyl alkoxysilane is, for example, methoxytrimethyl silane with the chemical formula (Me)₃Si(OMe) and the CAS number 1825-61-2, commercially available from Sigma-Aldrich.

Within the framework of the previously described embodiment, the color pigment that represents the colored core, can be treated with the surface treatment agent in a coating reaction, such that a simply coated pigment is obtained, which comprises the colored core and a silicon-containing coating. The silicon-containing coating in this case is the only and also the outer coating.

The silicon atoms (and/or the structural units, which comprise at least one silicon atom) present in the outer coating of the pigment can enter an interaction with the silanes applied on the keratin material in the step (b). It is presumed that ties, aggregates and/or agglomerates form due to these interactions. In this manner, the color pigments can be fixed on the keratin material in an especially sturdy and wash-proof film

Within the framework of a further very specifically preferred embodiment, it is also possible to coat the color pigment with multiple materials, so a multiple coated pigment can be used in the method as invented. This multiple coated pigment comprises a colored core, on this core at least one further coating and on this, at least one further coating the silicon-containing coating, which in turn represents the outer coating. As the multiple coated pigments also contain structure units with silicon atoms in their outer coating, they can also enter interaction in a similar manner with the silanes applied on the keratin material on application in step (b) in the method as invented. Particularly sturdy and washable films can also be generated on the keratin material in this manner on using multiple coated pigments.

In a further embodiment, a method is especially preferred, comprising the (a) use of an agent on the keratinous material, in which the agent contains at least one multiple coated pigment with a colored core and a silicon-containing outer coating.

For the manufacture of the multiple coated pigment, the use of polymers as coating materials has proven to be quite particularly good. The coating can in this case be effected with anionic polymers, cationic polymers and/or nonionic polymers.

In a further embodiment, a method is especially preferred, comprising the
(a) use of an agent on the keratinous material, in which the agent contains at least one multiple coated pigment that comprises the following layers:
(S1) optionally a coating with an anionic polymer
(S2) optionally a coating with a cationic polymer
(S3) optionally a coating with a nonionic polymer, and
(S4) a coating that is obtained by employing surface treatment with a surface treatment agent, in which the surface treatment agent is selected from the group of the tetra alkoxy silanes, alkyl trialkoxy silanes, the dialkyl dialkoxy silanes and the trialkyl alkoxy silanes,
and with the proviso, that the pigment possesses at least one of the coatings (S1), (S2) and/or (S3).

For the generation of the coating (S1), the use of an anionic polymer has proven to be quite particularly good.

For the generation of the coating (S2), the use of a cationic polymer has proven to be quite particularly good.

For the generation of the coating (S3), the use of a nonionic polymer has proven to be quite particularly good.

The sequence of the coating can be variously chosen—possible layer orders and for example (from inside to outside):
Colored core—first coating (S1) with anionic polymer, second and outer coating (S4) that is generated by treatment with tetra-alkoxy-silane as surface treatment agent,
colored core—first coating (S2) with cationic polymer, second and outer coating (S4) that is generated by treatment with tetra-alkoxy-silane as surface treatment agent,
colored core—first coating (S3) with nonionic polymer, second and outer coating (S4) that is generated by treatment with tetra-alkoxy-silane as surface treatment agent,
colored core—first coating (S1) with anionic polymer, second coating (S2) with a cationic polymer—third and outer coating (S4) that is generated by treatment with tetra-alkoxy-silane as surface treatment agent,
colored core—first coating (S1) with anionic polymer, second coating (S2) with a cationic polymer, third coating (S3) with a nonionic polymer, fourth and outer coating (S4) that is generated by treatment with tetra-alkoxy-silane as surface treatment agent,
Specifically intensive and washable coloration can be obtained, if a multiple coated pigment is used in the method as invented, which comprises four coatings (S1), (S2), (S3) and (S4).

In a further embodiment, a method is especially preferred, comprising the
(a) use of an agent on the keratinous material, in which the agent contains at least one multiple coated pigment that comprises the following layers:
(S1) a first coating with an anionic polymer,
(S2) a second coating with a cationic polymer,
(S3) a third coating with a nonionic polymer and

(S4) a fourth coating that is obtained by means of a surface treatment with a surface treatment agent, where the surface treatment agent is selected from the group of tetra alkoxy silanes, alkyl trialkoxy silanes, dialkyl dialkoxy silanes and of trialkyl alkoxy silanes.

In the framework of this quite specifically preferred embodiment, (S1) represents the first coating with an anionic polymer, which is present on the colored core (i.e., on the color pigment). The second coating (S2) with a cationic polymer is then applied on this first layer (S1). The third coating (S3) with a nonionic polymer is then applied on this second layer (S2). And now the surface treatment with the reactive surface treatment agent is performed on this third layer (S3), such that a cover or a coating is produced in an outer layer for a silicon-containing fourth coating.

The coating with an anionic polymer can be produced in the following manner, for example: The required quantity of an anionic polymer (for example 10.0 g) is dispersed or dissolved in 500 ml water under stirring at room temperature. Then the desired quantity (for example 10.0 g) of a color pigment is then added under continued stirring. This mixture is then ground for 60 minutes at room temperature in a ball mill (using ZrO2 balls, speed of rotation 3500 revolutions per minute). This suspension is then centrifuged three or four times for separating the superfluous anionic polymer and washed with distilled water.

The coating with a cationic or with a nonionic polymer can also proceed in a similar manner

Application of a second coating (S2) with cationic polymer on the first coating (S1) with anionic polymer can proceed, for example, as follows:

The previously obtained single coated pigments were redispersed in water (e.g., 10.0 g pigment in 500 ml water). A cationic polymer (e.g.,10.0 g) was then added to this aqueous solution with stirring. After 20 minutes, the cationic polymer precipitated in the form of a second coating on the first anionic coating. This suspension is then again centrifuged three or four times for separating the superfluous anionic polymer and then washed with distilled water.

The further coating with a non-ionic polymer and by employing surface treatment with a tetra alkoxy silane such as tetra ethoxy silane can now be performed as follows: 5.0 g of the previously coated pigment was dispersed in 35.0 g of water. This mixture was then diluted with 200 ml of ethanol. Thereafter, 200 mg polyvinyl pyrrolidone and 0.15 g of ammonia were added. Conditional on the addition of the nonionic polymer (polyvinyl pyrrolidone), a third coating (S3) was first produced with a nonionic polymer. Subsequently, the required quantity of tetra ethoxy silane (e.g., 5.0 g) in 40 ml ethanol were now added to this mixture within 60 minutes in small portions. The mixture was thereafter stirred for 24 hours at room temperature. To separate the coated pigments, they are again centrifuged three to four times, washed with distilled water and dried in vacuum for a few days at room temperature.

For the production of the coating (S1) with an anionic polymer, certain anionic polymers have proven to be quite particularly good.

Anionic polymers are such anionic polymers that feature carboxylate and/or sulfonate groups. Examples of anionic monomers, which such polymers could include, are acrylic acid, methacrylic acid, crotonic acid, maleic anhydride and 2-acrylamido-2-methyl propane sulfonic acid. In this case, the acidic groups can be present wholly or partially as sodium, potassium, ammonium, mono or triethanol ammonium salts. Preferred monomers are 2-acrylamido-2-methylpropane sulfonic acid and acrylic acid.

Anionic polymers containing 2-acrylamido-2-methylpropane sulfonic acid as sole or co-monomer, wherein the sulfonic acid group can be present wholly or partially as sodium, potassium, ammonium, mono or triethylammonium salt, have proved to be quite particularly effective.

Especially preferred is the homopolymer of the 2-acrylamido-2-methyl propane sulfonic acid, which is commercially available, for example under the name Rheothik® 11-80.

Within this embodiment, it can be preferred to use copolymers from at least one anionic monomer and at least one nonionic monomer. Regarding the anionic monomers, please refer to the above listed substances. Preferred nonionic monomers are acrylamide, methacrylamide, acrylic acid ester, methacrylic acid ester, vinyl pyrrolidone, vinyl ether and vinyl ester.

Preferred anionic copolymers are acrylic acid acrylamide copolymers as well as especially polyacrylamide copolymers with sulfonic acid group-containing monomers. A specially preferred anionic copolymer consists of 70 to 55 mol.% acrylamide and 30 to 45 mil.% acrylamido-2-methyl propane sulfonic acid, in which case the sulfonic acid group is present wholly or partially as sodium, potassium. Ammonium, mono or triethanol ammonium salt. This copolymer can also be present as cross-linked, in which case polyolefinic unsaturated compounds such as tetra allyloxythane, allylsucrose, allylpentaerythrite and methylene bisacrylamide can be preferably used as cross-linking agents. The commercial product Sepiegel®305 of the company SEPPIC contains such a polymer. The use of this compound, which contains, besides the polymer component, a hydrocarbon mixture (C₁₃-C₁₄-Isoparaffin) and a nonionogenic emulsifier (Laureth-7) has proved to be particularly advantageous within the framework of the current teachings.

The sodium acryloyldimethyltaurate copolymers sold under the name Simulgel®600 as compound with isohexadecane and polysorbate-80 have proved to be particularly effective.

Similarly preferred anionic homopolymers are non-crosslinked and crosslinked polyacrylic acids. Allyl ethers of pentaerythritol, of sucrose, or of propylene can be preferred crosslinking agents in such cases. Such compounds are commercially available, for example, under the trademark Carbopol®.

Copolymers from maleic anhydride and methyl vinyl ether, especially those with crosslinking, are also color-preserving polymers. A maleic acid methyl vinyl ether copolymer crosslinked with 1.9-decadiene is available commercially under the name Stabileze® QM.

Quite specifically preferred anionic polymers can be selected from the group including homo and copolymers of the styrene-4-sulfonic acid, the homo and copolymers of acrylic acid, the homo and copolymers of methacrylic acid, the homo and copolymers of crotonic acid, the homo and copolymers of maleic acid, the homo and copolymers of the 2-acrylamido-2-methyl propane sulfonic acid and/or their physiologically compatible salts.

In a further quite particularly preferred embodiment, a method is characterized in that the coated pigment comprises

(S1) a coating with an anionic polymer from the group consisting of homo and copolymers of the styrene-4-sulfonic acid, of the homo and copolymers of acrylic acid, of the homo and copolymers of methacrylic acid, of the homo and copolymers of crotonic acid, of the homo and copolymers of maleic acid, of the homo and copolymers of the 2-acrylamido-2-methyl propane sulfonic acid and their physiologically compatible salts.

As homopolymers of the styrene-4-sulfonic acid, for example, poly (4-styrene sulfonic acid) and/or their sodium salt can be used.

For the production of the coating (S2) with a cationic polymer, certain cationic polymers have proven to be quite particularly good.

Cationic polymers are to be understood as those, which feature a group in their main and/or side chain, which can be temporarily or permanently cationic. As contemplated herein, “permanently cationic” designates polymers that feature a cationic group irrespective of the pH value of the agent. They are generally polymers that contain a quaternary nitrogen atom, for example in the form of an ammonium group. Preferred cationic groups are quaternary ammonium groups. Especially such polymers in whose case the quaternary ammonium group is bound to a polymer main chain formed from acrylic acid, methacrylic acid or their derivatives via a C1-4 hydrocarbon group, have proved themselves to be especially suitable.

Other cationic polymers as contemplated herein are what are called “temporarily cationic” polymers. These polymers usually contain an amino group, which exists as quaternary ammonium group at certain pH values and thus cationic.

The cationic polymers can be solidifying and/or film-forming and/or anti-static and/or avivating polymers as well as polymers with conditioning and/or thickening properties. Suitable cation active polymers are preferably solidifying and/or conditioning polymers. Polymers are to be taken to mean natural as well as synthetic polymers, which can be cationically or amphoterically charged.

These two groups of polymers have a potentially cationic charge in common. Cationic as well amphoteric or zwitterionic polymers can therefore be characterized through their cationic charge density. The polymers as contemplated herein excel due to a charge density of at least 1 to 7 meq/g. A charge density of at least 2 to 7 meq/g is preferred in this case Especially preferred is a charge density of at least equal to 3 meq/g to 7 meq/g.

A further characteristic feature of the polymers usable in the coating is their molar mass. Molar mass of the respective polymers is understood to be the molar mass which the manufacturer indicates in the corresponding data sheets as measured by their method. For the selection of a suitable polymer, a molar mass of at least 50,000 g/u is seen to be suitable. Polymers with a molar mass of more than 100,000 g/u have proved to be especially suitable. Polymers with a molar mass of more than 1,000,000 g/u have proved to be especially suitable.

The cationic polymers can be homo- or copolymers or polymers, in which case the quaternary nitrogen groups are contained either in the polymer chain or preferably as substituents on one or more of the monomers. The monomers containing ammonium groups can be co-polymerized with non-cationic monomers. Suitable cationic monomers are unsaturated, radically polymerizable compounds which carry at least one cationic group, in particular ammonium-substituted vinyl monomers such as trialkylmethacryloxyalkyl ammonium, trialkylacryloxyalkyl ammonium, dialkyldiallyl ammonium and quaternary vinylammonium monomers with cyclic groups containing cationic nitrogen, such as pyridinium, imidazolium or quaternary pyrrolidones, e.g., alkylvinylimidazolium, alkylvinyl pyridinium, or alkylvinyl pyrrolidone salts. The alkyl groups of these monomers are preferably lower alkyl groups such as C1 to C7 alkyl groups, especially preferably C1 to C3 alkyl groups.

The monomers containing ammonium groups may be copolymerized with non-cationic monomers. Suitable co-monomers are for example acrylamide, methacrylamide; alkyl and dialkyl acrylamide, alkyl and dialkyl methacrylamide, alkyl acrylate, alkyl methacrylate, vinyl caprolactone, vinyl caprolactam, vinyl pyrrolidone, vinyl esters, z. B. vinyl acetate, vinyl alcohol, propylene glycol or ethylene glycol, the alkyl groups of these monomers preferably being C1 to C7 alkyl groups, particularly preferably C1 to C3 alkyl groups.

Especially preferred cationic polymers are homopolymers of the general formula (P1),

—{CH₂—[CR¹COO—(CH₂)ₘN⁺R²R³R⁴]}ₙX⁻ (P1)

Where R¹=—H or —CH₃
R², R³ and R⁴ are selected independently of each other from C1-4-alkyl-, -alkenyl- or -hydroxyalkyl groups,
m=1, 2, 3 or 4,
n is a natural number and
X⁻ is a physiologically compatible organic or inorganic anion, as well as copolymers, consisting predominantly of the monomer units as well as nonionogenic monomer units listed in the formula.
Within the framework of these polymers, preference is given to those for which at least one of the following conditions applies:
   R¹ stands for a methyl group
   R², R³ and R⁴ stand for methyl groups
   m has the value 2.

Examples of physiologically compatible counterions X that can be considered are halogenide ions, sulfate ions, phosphate ions, methosulfate ions as well as organic ions such as lactate, citrate, tartrate and acetate ions. Preferred are halogenide ions, especially chloride.

A particularly suitable homopolymer is poly (methacryloyloxyethyltrimethylammonium chloride) with the INCI designation polyquaternium-37, which is cross-linked if desired. Such products are commercially available, for example under the names Rheocare® CTH (Cosmetic Rheologies) and Synthalen® CR (3V Sigma). The cross-linking can be effected, if desired, by employing multiple olefinic unsaturated compounds, such as divinyl benzol, tetraallyloxy ethane, methylenbisacryl amide, diallyl ether, polyallylpolyglyceryl ether, or allyl ethers of sugars or sugar derivatives such as erythritol, pentaerythritol, arabitol, mannitol, sorbitol, sucrose or glucose. Methylenebisacrylamide is a preferred cross-linking agent.

The homopolymer is preferably used in the form of a non-aqueous polymer dispersion that should not have a polymer component below 30 weight %. Such polymer dispersions are commercially available under the names Salcare® SC 95 (about 50% polymer component, other components: Mineral oil (INCI designation: Mineral Oil) and tridecyl-polyoxypropylene-polyoxyethylene-ether (INCI-designation: PPG-1-Trideceth-6)) and Salcare® SC 96 (about 50% polymer component, other components: Mixture of diesters of propylene glycol with a mixture of capryl- and caprinic acid (INCI-designation: propylene glycol dicaprylate/dicaprate) and tridecyl-polyoxypropylen-polyoxyethylen-ether (INCI-designation: PPG-1-Trideceth-6)).

Suitable copolymers contain non-ionogenic monomer units besides a permanent or temporary cationic monomer. Preferred nonionic monomer units are acryl amide, methacrylamide, acrylic acid C1-4-alkylester and methacrylic acid-C1-4-alkylester. Out of these nonionogenic monomers, acryl amide is particularly preferred. These copolymers can also be cross-linked, as in the case of the homopolymers described above. A preferred copolymer is the cross-linked acryl amide methacyloyloxyethyl trimethyl ammonium chloride copolymer. Such copolymers, in which the monomers are present in a weight ratio of about 20:80, are commercially available as about 50% non-aqueous polymer dispersion under the name Salcare® SC 92.

Quite specifically preferred cationic polymers can be selected from the group of homo and copolymers of dimethyldiallyl ammonium salts, of the homo- and copolymers of tri-C₁-C₆-alkyl-methacryloxy-C₁-C₆-alkyl-ammonium salts, of the homo- and copolymers of tri-C₁-C₆-alkyl-acryloxy-C₁-C₆-alkylammonium salts and of the homo- and copolymers of 1-vinyl-3-(C₁-C₆-alkyl) imidazolium salts.

In a further quite particularly preferred embodiment, a method as invented is characterized in that the coated pigment comprises.

(S2) a coating with a cationic polymer from the group of homo and copolymers of dimethyl diallyl ammonium salts, of the homo- and copolymers of tri-C₁-C₆-alkyl-methacryloxy-C₁-C₆-alkyl-ammonium salts, of the homo- and copolymers of tri-C₁-C₆-alkyl-acryloxy-C₁-C₆-alkyl ammonium salts and/or of the homo- and copolymers of 1-vinyl-3-(C₁-C₆-alkyl) imidazolium salts.

An explicitly quite especially well-suited cationic polymer is the homopolymer of dimethyl diallyl ammonium chloride, which is also known under the INCI designation polyquaternium-6 and is commercially available under the trade name Merquat 100 from Lubrizol.

For the production of the coating (S3) with a nonionic polymer, certain cationic polymers have also proven to be quite particularly well-suited.

Suitable nonionic polymers are for example:
Vinylpyrrolidone/Vinyl ester copolymers, as they are sold under the trademark Luviskol® (BASF). Luviskol® VA 64 and Luviskol® VA 73, both vinylpyrrolidone/vinyl acetate copolymers, are also preferred non-ionic polymers.
Cellulose ethers, such as hydroxypropyl cellulose, hydroxyethyl cellulose and methylhydroxypropyl cellulose, marketed for example under the trademarks Culminal® and Benecel® (Aqualon) and Natrosol® types (Hercules).
Starches and their derivatives, especially starch ether, such as Structure® XL (National Starch), a multi-functional, salt-tolerant starch.
Shellac
Polyvinylpyrrolidone, as they are sold under the designation Luviskol® (BASF).
Siloxane: These siloxanes can be both water-soluble and water insoluble. Both volatile and non-volatile siloxanes are suitable. Non-volatile siloxanes are compounds whose boiling point at normal pressure is above 200° C. Preferred siloxanes are polydialkylsiloxanes, such as polydimethylsiloxane, polyalkylarylsiloxanes, such as polyphenylmethylsiloxane, ethoxylated polydialkylsiloxanes and polydialkylsiloxanes containing amine and/or hydroxy groups.
Glycosidically substituted silicone.

Quite specifically preferred nonionic polymers can be selected from the group of the homo and copolymers of vinyl pyrrolidones, of the homo and copolymers vinyl acetate, of the homo and copolymers of styrenes, of the homo- and copolymers of ethenes and/or of homo and copolymers of vinyl alcohol.

In a further quite particularly preferred embodiment, a method as invented is characterized in that the coated pigment comprises.

(S3) a coating with a nonionic polymer from the group of the homo and copolymers of vinyl pyrrolidones, of the homo- and copolymers vinyl acetate, of the homo- and copolymers of styrenes, of the homo- and copolymers of ethene and/or of homo- and copolymers of vinyl alcohol.

Depending on the required color intensity, the agents used in the step (a) of the process can contain a differing content of one or multiple coated pigments. The more intensive the desired color result, the higher will be the quantity to be used as chosen by the expert. Usually the agent applied in step (a) can contain—in relation to its total weight—one or more coated pigments in a total quantity of about 0.01 to about 10.0% by weight, preferably from about 0.1 to about 8.0% by weight, further preferably from about 0.2 to about 6.0% by weight and quite specifically preferred from about 0.5 to about 4.5% by weight.

In a further preferred embodiment, a method as invented is characterized in that the agent used in step (a) contains—based on its total weight—one or more coated pigments in a total amount of from about 0.01 to about 10.0% by weight, preferably from about 0.1 to about 8.0% by weight, more preferably from about 0.2 to about 6.0% by weight and quite particularly preferably from about 0.5 to about 4.5% by weight.

Silanes with One, Two or Three Silicon Atoms

The step (b) of the method as invented comprises the use of an agent on the keratinous material, wherein the agent contains at least one organic silicon compound from the group of silanes with one, two or three silicon atoms.

The silanes applied in step (b) of the method as invented are silanes that are not present as absorbed in a pigment. So, the silicon compounds used in the steps (a) and (b) are different from each other.

Particularly preferably, the step (b) of the method comprises the use of an agent on the keratinous material, in which the agent comprises at least one organic silicon compound selected from silanes having one, two or three silicon atoms, wherein the organic silicon compound comprises one or more hydroxyl groups or hydrolyzable groups per molecule.

These organic silicon compounds or organic silanes used in step (b) of the method are reactive compounds.

Organic silicon compounds, alternatively called organosilicon compounds, are compounds which either have a direct silicon-carbon bond (Si-C) or in which the carbon is bonded to the silicon atom via an oxygen, nitrogen or sulfur atom. The organic silicon compounds as contemplated herein are compounds containing one to three silicon atoms. Organic silicon compounds preferably contain one or two silicon atoms.

According to IUPAC rules, the term silane stands for a group of chemical compounds based on a silicon skeleton and hydrogen . In organic silanes, the hydrogen atoms are completely or partially replaced by organic groups such as (substituted) alkyl groups and/or alkoxy groups. In organic silanes, some of the hydrogen atoms may also be replaced by hydroxy groups.

In a particularly preferred embodiment, a method as contemplated herein is exemplified by the application of an agent (a) to the keratinous material, said agent (a) comprising at least one organic silicon compound selected from silanes having one, two or three silicon atoms, said organic silicon compound further comprising one or more hydroxyl groups or hydrolyzable groups per molecule.

In a particularly preferred embodiment, a method as contemplated herein is exemplified by the application of an agent (a) to the keratinous material, said agent (a) comprising at least one organic silicon compound selected from silanes having one, two or three silicon atoms, said organic silicon compound further comprising one or more basic chemical functions and one or more hydroxyl groups or hydrolyzable groups per molecule.

This basic group can be, for example, an amino group, an alkylamino group or a dialkylamino group, which is preferably connected to a silicon atom via a linker. The basic group is preferably an amino group, a C₁-C₆ alkylamino group or a di(C₁-C₆) alkylamino group.

The hydrolyzable group(s) is (are) preferably a C₁-C₆ alkoxy group, especially an ethoxy group or a methoxy group. It is preferred when the hydrolyzable group is directly bonded to the silicon atom. For example, if the hydrolyzable group is an ethoxy group, the organic silicon compound preferably contains a structural unit R′R″R′″Si—O—CH2—CH3. The residues R′, R″ and R′″ represent the three remaining free valences of the silicon atom.

A quite particularly preferred method as contemplated herein is exemplified in that at least one organic silicon compound selected from silanes having one, two or three silicon atoms, is used on the keratinous material in step (b), the organic silicon compound preferably comprising one or more basic chemical functions and one or more hydroxyl groups or hydrolyzable groups per molecule.

Particularly good results could be obtained if the agent of the step (b) as invented contains at least one organic silicon compound of formula (I) and/or (II).

The compounds of formulas (I) and (II) are organic silicon compounds selected from silanes having one, two or three silicon atoms, the organic silicon compound comprising one or more hydroxyl groups and/or hydrolysable groups per molecule.

In a further embodiment, a method is especially preferred, comprising the (b) use of an agent on the keratinous material, where the agent contains at least one organic silicon compound of the formula (I) and/or (II)

R₁R₂N-L-Si(OR₃)ₐ(R₄)_{b} (I),
where
R₁, R₂ independently represent a hydrogen atom or a C₁-C₆ alkyl group,
L is a linear or branched divalent C₁-C₂₀ alkylene group,
R₃ represents a hydrogen atom or a C₁-C₆ alkyl group,
R₄ represents a C₁-C6 alkyl group
   a, stands for an integer from 1 to 3, and
   b stands for the integer 3−a,

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
where
R5, R5′, R5″ independently represent a hydrogen atom or a C₁-C6 alkyl group,
R6, R6′ and R6″ independently represent a C₁-C₆ alkyl group,
A, A′, A″, independently of one another represent a linear or branched divalent C₁-C₂₀ alkylene group
R₇ and R₈ independently represent a hydrogen atom, a C₁-C₆ alkyl group, a hydroxy C₁-C₆ alkyl group, a C₂-C₆ alkenyl group, an amino C₁-C6 alkyl group or a group of formula (III)

-(A″″)-Si(R6″)_{d}″(OR₅″)_{c″} (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
provided that at least one of e, f, g and h is different from 0.

The substituents R₁, R₂, R₃, R₄, R₅, R₅′, R₅″, R₆, R₆′, R₆″, R_{7,} R₈, L, A′, A″″ and A″″ in the compounds of formula (I) and (II) are explained below as examples:

Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂),), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. Quite particularly preferred, the radicals R₁ and R₂ both represent a hydrogen atom.

In the middle part of the organic silicon compound is the structural unit or the linker -L- which stands for a linear or branched, divalent C₁-C₂₀ alkylene group.

A bivalent C₁-C₂₀-alkylene group can alternatively also be designated as a divalent or dual bonded C₁-C₂₀-alkylene group, which means that each grouping L can accept two bonds. A bond is created from the amino group R1R2N to the linker L and the second bond is created between the linker L and the silicon atom.

Preferably -L- stands for a linear, divalent C₁-C₂₀ alkylene group. Further preferably -L- stands for a linear divalent C₁-C₆ alkylene group. Particularly preferred -L stands for a methylene group (CH₂—), an ethylene group (—CH₂—CH₂—), propylene group (—CH₂—CH₂—CH₂—) or butylene (—CH₂—CH₂—CH₂—CH₂—). Quite particularly preferably, L stands for a propylene group (—CH₂—CH₂—CH₂—)

The linear propylene group (—CH₂—CH₂—CH₂—) can alternatively also be designated as propane-1,3-diyl group.

The organic silicon compounds of formula (I)

R₁R₂N-L-Si(OR₃)ₐ(R₄)_{b} (I),

one end of each carries the silicon-containing group —Si(OR₃)ₐ(R₄)_{b}

In the terminal structural unit —Si(OR₃)ₐ(R₄)_{b}, R₃ is hydrogen or C₁-C₆ alkyl group, and R₄ is C₁-C₆ alkyl group. Particularly preferred, R₃ and R₄ independently of each other represent a methyl group or an ethyl group.

Here a stands for an integer from 1 to 3, and b stands for the integer 3−a. If a stands for the number 3, then b is equal to 0. If a stands for the number 2, then b is equal to 1. If a stands for the number 1, then b is equal to 2.

Particularly wash fast films could be obtained if the agent of step (b) contains at least one organic silicon compound corresponding to formula (I): in which the radicals R₃, R₄ independently of one another represent a methyl group or an ethyl group.

On appropriately using the method as invented for dyeing keratinous material, dyes with the best wash fastness values could be obtained if the agent of the step (b) contains at least one organic silicon compound corresponding to formula (I): in which the radicals R₃, R₄ independently of one another represent a methyl group or an ethyl group.

Furthermore, coloring with the best wash fastness properties could be obtained if the agent as contemplated herein contains at least one organic silicon compound of formula (I) in which the radical a represents the number 3. In this case the rest b stands for the number 0.

A further especially preferred embodiment features a method comprising the (b) use of an agent on the keratinous material, wherein the agent contains at least one organic silicon compound of the formula (I), in which
R3, R4 independently of one another represent a methyl group or an ethyl group and
a stands for the number 3 and
b stands for the number 0.
A further especially preferred embodiment features a method comprising the
(b) use of an agent on the keratinous material, where the agent contains at least one organic silicon compound of the formula (I) and

R₁R₂N-L-Si(OR₃)ₐ(R₄)_{b} (I),

where
R₁, R₂ both represent a hydrogen atom, and
L represents a linear, divalent C₁-C₆-alkylene group, preferably a propylene group (—CH₂—CH₂—CH₂—) or an ethylene group (—CH₂—CH₂—),
R₃ represents a hydrogen atom, an ethyl group or a methyl group,
R₄ represents a methyl group or an ethyl group,
a stands for the number 3 and
b stands for the number 0.

Organic silicon compounds of the formula (I) which are particularly suitable for solving the problem as contemplated herein are

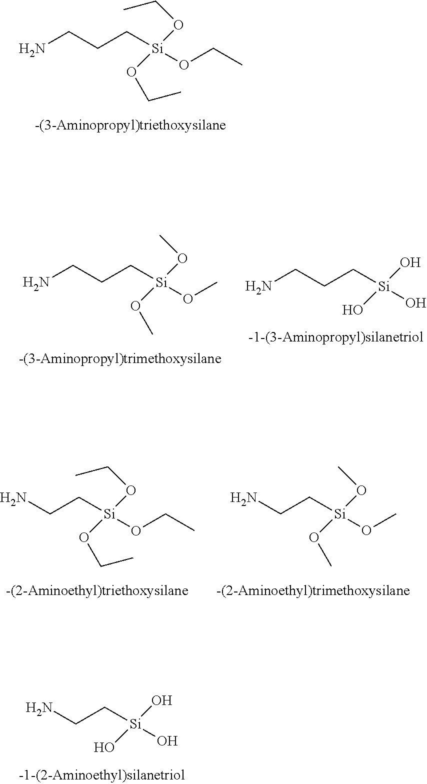

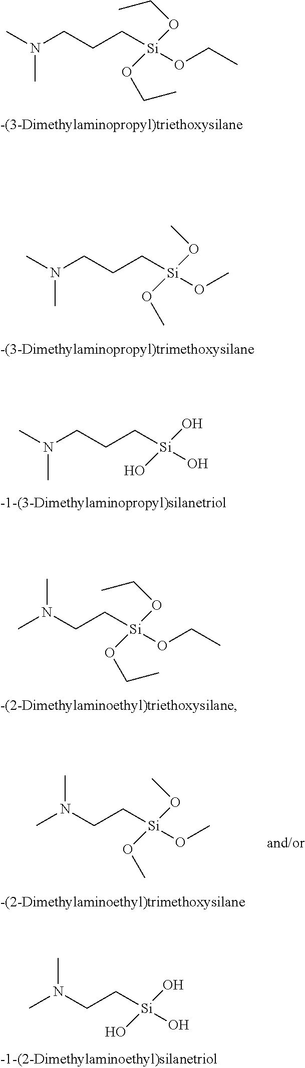

A further especially preferred embodiment features a method comprising the
(b) use of an agent on the keratinous material, where the agent contains at least one organic silicon compound selected from the group consisting of
(3-Aminopropyl)triethoxysilane
(3-Aminopropyl)trimethoxysilane
1-(3-Aminopropyl)silanetriol
(2-Aminoethyl)triethoxysilane
(2-Aminoethyl)trimethoxysilane
1-(2-Aminoethyl)silanetriol
(3-Dimethylaminopropyl)triethoxysilane
(3-Dimethylaminopropyl)trimethoxysilane
1-(3-Dimethylaminopropyl)silanetriol
(2-Dimethylaminoethyl)triethoxysilane.
(2-Dimethylaminoethyl)trimethoxysilane and
1-(2-Dimethylaminoethyl)silanetriol.

The aforementioned organic silicon compounds of formula (I) are commercially available.

(3-aminopropyl)trimethoxysilane, for example, can be purchased from Sigma-Aldrich. Also (3-aminopropyl)triethoxysilane is commercially available from Sigma-Aldrich.

Within the framework of a further embodiment, the agent used in the step (b) of the method as invented contains at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II).

The organosilicon compounds of formula (II) as contemplated herein each carry the silicon-containing groups (R₅O)_{c}(R₆)_{d}Si— and —Si(R₆′)_{d′}(OR₅′)_{c} at both ends.

In the central part of the molecule of formula (II) there are the groups -(A)ₑ- and —[NR₇-(A′)]_{f}-

and —[O-(A″)]_{g}- and —[NR₈-(A′″)]ₕ-. Here, each of the radicals e, f, g and h can independently of one another stand for the number 0 or 1, with the proviso that at least one of the radicals e, f, g and h is different from 0. In other words, an organic silicon compound of formula (II) as contemplated herein contains at least one grouping from the group including -(A)- and —[NR₇-(A′)]- and —[O-(A″)]- and —[NR₈-(A″′)]-.

In the two terminal structural units (R₅O)_{c}(R₆)_{d}Si— and —Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from 1 to 3, and d stands for the integer 3−c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3−c′. If c′ stands for the number 3, then d′ is 0. If c′ stands for the number 2, then d′ is 1. If c′ stands for the number 1, then d′ is 2.

Films with the highest stability or dyes with the best wash fastness values could be obtained if the radicals c and c′ both stand for the number 3. In this case d and d′ both stand for the number 0.

A further especially preferred embodiment features a method comprising the (b) use of an agent on the keratinous material, where the agent contains at least one organic silicon compound of the formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
R5 and R5′ independently represent a methyl group or an ethyl group,
c and c′ both stand for the number 3 and
d and d′ both stand for the number 0.

If c and c′ are both the number 3 and d and d′ are both the number 0, the organic silicon compound of the present disclosure corresponds to formula (IIa)

(R₅O)₃Si-(A)ₑ-[NR₇-(A′)]_{f}-[O-(A″)]_{g}-[NR₈-(A′″)]ₕ-Si(OR₅′)₃ (IIa).

The radicals e, f, g and h can independently stand for the number 0 or 1, whereby at least one radical from e, f, g and h is different from zero. The abbreviations e, f, g and h thus define which of the groupings -(A)ₑ- and —[NR7-(A′)]_{f}- and —[O-(A″)]_{g} and —[NR8-(A″′)]ₕ- are located in the middle part of the organic silicon compound of formula (II).

In this context, the presence of certain groupings has proved to be particularly beneficial in terms of increasing washability. Particularly good results were obtained when at least two of the residues e, f, g and h stand for the number 1. Especially preferred e and f both stand for the number 1. Furthermore, g and h both stand for the number 0.

If e and f both stand for the number 1 and g and h both stand for the number 0, the organic silicon compound as contemplated herein corresponds to formula (IIb)

(R₅O)_{c}(R₆)_{d}Si-(A)-[NR₇-(A′)]-Si(R₆′)_{d′}(OR₅′)ₑ (IIb).

The radicals A, A′, A″, A″′ and A″″ independently represent a linear or branched divalent C₁-C₂₀ alkylene group. Preferably the A, A′, A″, A″′ and A″″ independently of one another represent a linear, divalent C₁-C₂₀ alkylene group. Further preferably the radicals A, A′, A″, A″′ and A″″ independently represent a linear divalent C₁-C₆ alkylene group. In particular, the radicals A, A′, A″, A″′ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). Quite particularly preferably, the radicals A, A′, A″, A″′ and A″″ stand for a propylene group (—CH₂—CH₂—CH₂—).

A bivalent C₁-C₂₀-alkylene group can alternatively also be designated as a divalent or dual bonded C₁-C₂₀-alkylene group, which means that each grouping A, A′, A″, A″′ and A″″ can accept two bonds.

The linear propylene group (—CH₂—CH₂—CH₂—) can alternatively also be designated as propane-1,3-diyl group.

If the radical f represents the number 1, then the organic silicon compound of formula (II) as contemplated herein contains a structural grouping —[NR₇-(A′)]-. If the radical f represents the number 1, then the organic silicon compound of formula (II) as contemplated herein contains a structural grouping —[NR₈-(A″′)]-.

In this case, the radicals R7 and Rs independently represent a hydrogen atom, a C₁-C₆-alkyl group, a hydroxy-C₁-C₆-alkyl group, a C₂-C₆-alkenyl group, an amino-C₁-C₆-alkyl group or a group of the formula (III)

-(A″″)-Si(R₆″)_{d}″(OR₅″)_{c″} (III).

Very preferably the radicals R7 and R8 independently of one another represent a hydrogen atom, a methyl group, a 2-hydroxyethyl group, a 2-alkenyl group, a 2-aminoethyl group or a grouping of the formula (III).

If the radical f represents the number 1 and the radical h represents the number 0, the organic silicon compound as contemplated herein contains the grouping [NR₇-(A′)] but not the grouping —[NR₈-(A″′)]. If the radical R7 now stands for a grouping of the formula (III), the agent (a) contains an organic silicone compound with 3 reactive silane groups.

A further preferred embodiment features a method comprising in step

(b) the use of an agent on the keratinous material, where the agent contains at least one organic silicon compound of the formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
e and f both stand for the number 1,
g and h both stand for the number 0,
A and A′ independently represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

In a further preferred embodiment, a process as contemplated herein is exemplified in that the agent applied in step (a) contains at least one organic silicon compound of the formula (II), wherein
e and f both stand for the number 1,
g and h both stand for the number 0,
A and A′ independently of one another represent a methylene group(—CH₂—), an ethylene group (—CH₂—CH₂—) or a propylene group (—CH₂—CH₂—CH₂), and
R7 represents a hydrogen atom, a methyl group, a 2-hydroxyethyl group, a 2-alkenyl group, a 2-aminoethyl group or a group of formula (III).

Organic silicon compounds of the formula (II) which are well suited for solving the problem as contemplated herein are

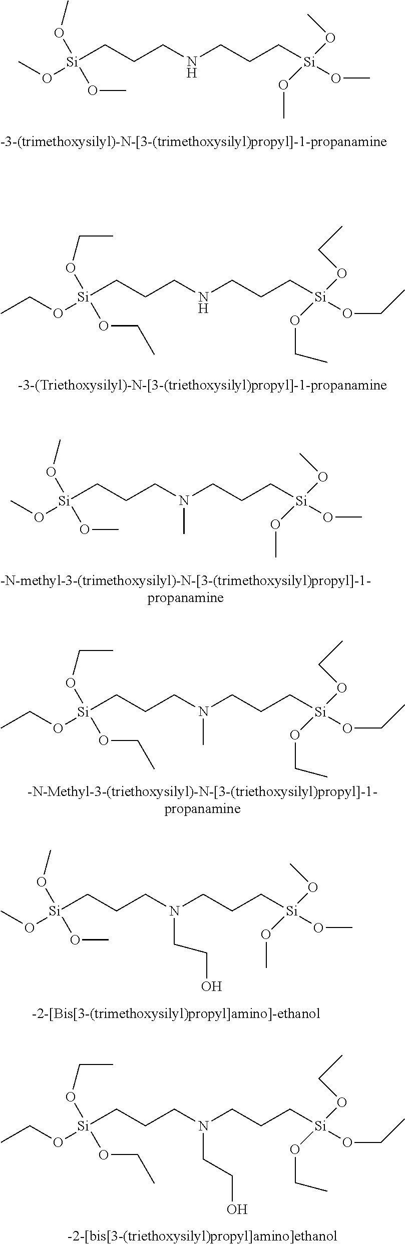

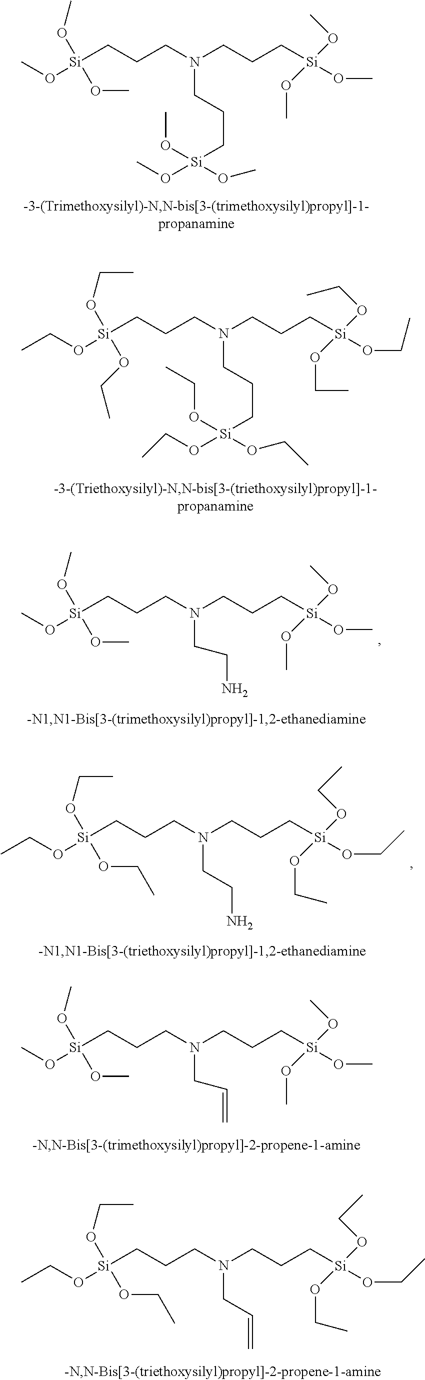

The aforementioned organic silicon compounds of formula (II) are commercially available.

Bis(trimethoxysilylpropyl)amines with the CAS number 82985-35-1 can be purchased from Sigma-Aldrich.

Bis[3-(triethoxysilyl)propyl]amines with the CAS number 13497-18-2 can be purchased from Sigma-Aldrich, for example.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

A further preferred embodiment features a method comprising in step
(b) the use of an agent on the keratinous material, where the agent contains at least one organic silicon compound selected from the group including
3-(trimethoxysilyl)-N-[3-(trimethoxysilyl) propyl]-1-propane amine
3-(Triethoxysilyl)-N-[3-(triethoxysilyl) propyl]-1-propanamine
N-methyl-3-(trimethoxysilyl)-N-[3-(trimethoxysilyl) propyl]-1-propanamine
N-Methyl-3-(triethoxysilyl)-N-[3-(triethoxysilyl) propyl]-1-propanamine
2-[Bis[3-(trimethoxysilyl) propyl]amino]-ethanol
2-[bis[3-(triethoxysilyl) propyl]amino]ethanol
3-(Trimethoxysilyl)-N,N-bis[3-(trimethoxysilyl) propyl]-1-propanamine
3-(Triethoxysilyl)-N,N-bis[3-(triethoxysilyl) propyl]-1-propanamine
N1,N1-bis[3-(trimethoxysilyl) propyl]-1,2-ethanediamine,
N1,N1-bis[3 (triethoxysilyl) propyl]-1,2-ethanediamine,
N,N-bis[3 (trimethoxysilyl)propyl]-2-propene-1-amine and/or
N,N-bis[3 (triethoxysilyl)propyl]-2-propene-1-amine

In further tests, especially dyeing tests, it has also proved to be particularly advantageous if the agent used on the keratinous material in step (b) in the method as invented contains at least one organic silicon compound of formula (IV)

R₉Si(OR₁₀)ₖ(R₁₁)ₘ (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be called a silane of the alkyl-alkoxy-silane or alkyl-hydroxy-silane type,

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In a further preferred embodiment, a process is preferred, comprising in step (b) the use of an agent on the keratinous material where the agent contains at least one organic silicon compound of the formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

A further preferred embodiment features a method comprising in step

(b) the use of an agent on the keratinous material, wherein the agent contains, in addition to the organic silicon compound(s) of formula (II), at least one more organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

A further preferred embodiment features a method comprising in step

(b) the use of an agent on the keratinous material, wherein the agent contains, in addition to the organic silicon compound(s) of formula (II), at least one more organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

A further preferred embodiment features a method comprising in step

(b) the use of an agent on the keratinous material, wherein the agent contains, in addition to the organic silicon compound(s) of formula (I) and/or (II), at least one more organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In the organic silicon compounds of formula (IV), the radical R₉ represents a C₁-C₁₂ alkyl group. This C₁-C₁₂ alkyl group is saturated and can be linear or branched. Preferably R₉ stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferred, R₉ stands for a methyl group, an ethyl group or an n-octyl group.

In the organic silicon compounds of formula (IV), the radical R10 represents a hydrogen atom or a C1-C6 alkyl group. In particular, R10 stands for a methyl group or an ethyl group.

In the organic silicon compounds of formula (IV), the radical R₁₁ represents a C₁-C₆ alkyl group. In particular, R11 stands for a methyl group or an ethyl group.

Furthermore, k stands for a whole number from 1 to 3, and m stands for the whole number 3−k. If k stands for the number 3, then m is equal to 0. If k stands for the number 2, then m is equal to 1. If k stands for the number 1, then m is equal to 2.

Particularly sturdy films, i.e., dyes with particularly good wash fastness values could be obtained if an agent was used in the process in step (b), which contains at least one organic silicon compound of the formula (IV) in which the radical k stands for the number 3. In this case the rest m stands for the number 0.

Organic silicon compounds of the formula (IV) which are particularly suitable for solving the problem as contemplated herein are

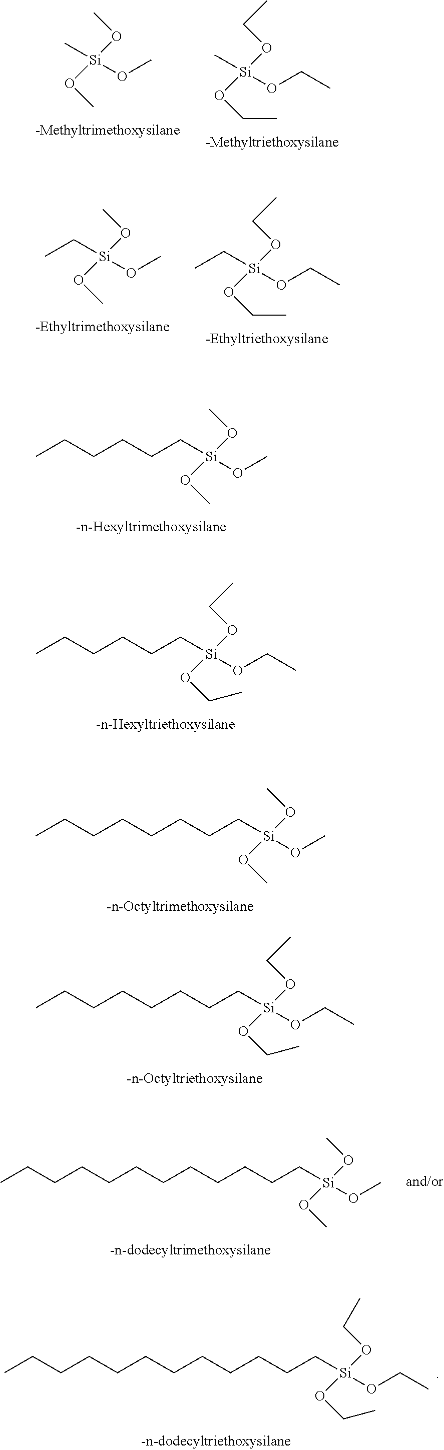

A further preferred embodiment features a method comprising in step

(b) the use of an agent on the keratinous material, where the agent contains at least one organic silicon compound of the formula (IV), which is selected from the group including
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane and/or
Dodecyltriethoxysilane.

The organic silicon compounds described above are reactive compounds. In this context, it has been found to be preferable if the agent used in the step (b)—based on the total weight of agent—contains one or more organic silicon compounds in a total amount of about 0.1 to about 20.0% by weight, preferably from about 1.0 to about 15.0% by weight and particularly preferably from about 2.0 to about 8.0% by weight.

In a further preferred embodiment, a process as contemplated herein is exemplified in that the agent used in step (b)—based on the total weight of agent (a)—contains one or more organic silicon compounds in a total amount of about 0.1 to about 20.0% by weight, preferably from about 1.0 to about 15.0% by weight and particularly preferably from about 2.0 to about 8.0% by weight.

To achieve particularly good dyeing results, it is particularly advantageous to use the organic silicon compounds of the formula (I) and/or (II) in certain quantity ranges in the agent of the step (b) of the method. Especially preferably, the agent contains—based on the total weight of the agent—one or more organic silicon compounds of the formula (I) and/or (II) in a total quantity of about 0.1 to about 10.0% by weight, preferably from about 0.5 to about 5.0% by weight and especially preferred from about 0.5 to about 3.0% by weight.

A further preferred embodiment features a method comprising in step (b) the use of an agent on the keratinous material, wherein the agent contains, in relation to the total weight of the agent, one or more organic silicon compounds of the formula (I) and/or (II) in a total quantity of about 0.1 to about 10.0% by weight, preferably from about 0.5 to about 5.0% by weight and particularly preferably from about 0.5 to about 3.0% by weight.

Furthermore, it has proven to be particularly preferred if the organic silicon compound(s) of formula (IV) is (are) also present in certain quantity ranges in the agent. In a further preferred embodiment, a method is preferred, comprising in step (b) the use of an agent on the keratinous material, wherein the agent contains, in relation to the total weight of the agent, one or more organic silicon compounds of the formula (IV) in a total quantity of about 0.1 to about 20.0% by weight, preferably from about 2.0 to about 15.0% by weight and particularly preferably from about 4.0 to about 9.0% by weight.

A further preferred embodiment features a method comprising in step (b) the use of an agent on the keratinous material, wherein the agent contains, in relation to the total weight of the agent, one or more organic silicon compounds of the formula or (IV) in a total quantity of about 0.1 to about 20.0% by weight, preferably from about 2.0 to about 15.0% by weight and particularly preferably from about 3.2 to about 10.0% by weight.

In the course of the work leading to this present disclosure it turned out that particularly sturdy and uniform films could be obtained on the keratin material if the agent used in the step (b) of the method contains two structurally different organic silicon compounds.

A further preferred embodiment features a method comprising in step (b) the use of an agent on the keratinous material. where the agent contains at least two structurally mutually different organic silicon compounds.

In a further quite especially preferred embodiment, a method is preferred, comprising in step (b) the use of an agent on the keratinous material, wherein the agent contains at least one organic silicon compound of the formula (I), which is selected from the group including (3-amino propyl) triethoxy silane and (3-aminopropyl) trimethoxy silane and. Additionally contains at least one organic silicon compound of the formula (IV), which is selected from the group of methyltrimethoxy silane, methyltriethoxy silane, ethyltrimethoxy silane and ethyl triethoxy silane.

A further preferred embodiment features a method comprising in step (b) the use of an agent on the keratinous material. where the agent contains, in relation to the total weight of the agent:
From about 0.5 to about 5.0 weight% of at least one first organic silicon compound selected from the group of (3-Aminopropyl)trimethoxysilane, (3-Aminopropyl)triethoxysilane, (2-Aminoethyl)trimethoxysilane, (2-Aminoethyl)triethoxysilane, (3-Dimethylaminopropyl)trimethoxysilane, (3-Dimethylaminopropyl)triethoxysilane (2-Dimethylaminoethyl)trimethoxysilane and (2-Dimethylaminoethyl)triethoxysilane, and
From about 3.2 to about 10.0% by weight of at least one second organic silicon compound selected from the group of methyltrimethoxysilane, methyltriethoxysilane, ethyltrimethoxysilane, ethyltriethoxysilane, octyltrimethoxysilane, octyltriethoxysilane, dodecyltrimethoxysilane and dodecyltriethoxysilane.

### Steps (a) and (b) of the Method

The method as invented is exemplified by its steps (a) and (b) i.e., by
(a) use of an agent on the keratinous material, in which the agent contains at least one coated pigment with a colored core and a silicon-containing coating, and
(b) use of an agent on the keratinous material, wherein the agent contains at least one organic silicon compound from the group of silanes with one, two or three silicon atoms.

The agent(s) used in the steps (a) and (b) are cosmetic products. Either the same agents can be used in the steps (a) and (b) or else different agents can be used steps (a) and (b). In addition, the steps (a) and (b) can take place either simultaneously or successively.

In a further preferred embodiment, a method as invented is exemplified in that the agents used in step (a) and in step (b) are the same or different.

In a further preferred embodiment, a method as invented is exemplified in that the agents used in step (a) and in step (b) can be applied simultaneously or successively on the keratinous material.

If the same agent is applied on the keratin material in the steps (a) and (b) of the method, both the steps take place simultaneously. In other words, within the framework of this embodiment, an agent is applied on the keratin material, which contains at least one coated pigment (a) as well as at least one organic silicon compound (b) from the group of silanes with one, two or three silicon atoms.

Within the framework of this embodiment, a particularly preferred method for dyeing keratinous material, in particular human hair, comprises the use of an agent on the keratinous material, wherein the agent contains
(a) at least one coated pigment with a colored core and a silicon-containing coating. and
(b) at least one organic silicon compound from the group of silanes having one, two or three silicon atoms.

Furthermore, it is similarly preferred if different agents are applied on the keratin material in the steps (a) and (b) of the method.

Within the framework of this further embodiment, there is a preferred process for dyeing keratinous material, in particular human hair, comprising the following steps:
(a) use of an agent on the keratinous material, in which the agent contains at least one coated pigment with a colored core and a silicon-containing coating, and
(b) use of an agent on the keratinous material, wherein the agent contains at least one organic silicon compound from the group of silanes with one, two or three silicon atoms, in which case the agents used in the steps (a) and (b) are different from each other.

The different agents are preferably used one after the other. In this case, the agent of the step (a) can be used first, followed by the use of the agent of the step (b). But the agent of the step (b) can also be used first, followed by the use of the agent of the step (a).

Within the framework of this further embodiment, there is a preferred process for dyeing keratinous material, in particular human hair, comprising the following steps in the stated order:
(1-a) use of an agent on the keratinous material, in which the agent contains at least one coated pigment with a colored core and a silicon-containing coating, and thereafter
(2-b) use of an agent on the keratinous material, wherein the agent contains at least one organic silicon compound from the group of silanes with one, two or three silicon atoms.

Explicitly quite particularly preferred is a method for dyeing keratinous material, in particular human hair, comprising the following steps in the stated order:
(1-b) use of an agent on the keratinous material, wherein the agent contains at least one organic silicon compound from the group of silanes with one, two or three silicon atoms, and thereafter
(2-a) use of an agent on the keratinous material, in which the agent contains at least one coated pigment with a colored core and a silicon-containing coating.

The agents used in the steps (a) and (b) of the method as invented are ready-to-use products. These are preferably aqueous, which means that the agent used in the step (a) of the method contains the coated pigment(s) in an aqueous or aqueous/alcoholic cosmetic carrier. This cosmetic carrier can be fluid, in gel form or in a cream form. For the purpose of hair coloration, such carriers are, for example, creams, emulsions, gels or surfactant-containing foaming solutions, such as shampoos, foam aerosols, foam formulations or other preparations suitable for application to the hair.

The agents used in step (b) of the method is preferably aqueous, so the organic silicon-containing compound from the group of silanes with one, two or three silicon atoms is also prepared in the form of an aqueous or aqueous/alcoholic cosmetic carrier.

The agent(s) used in step (a) and/or in step (b) of the method—based on their weight—preferably contain(s) at least 20% by weight, further preferably at least 30% by weight, yet more preferably at least 40% by weight and especially preferably at least 50% by weight of water. The cosmetic carrier can also be aqueous-alcoholic. Aqueous/alcoholic solutions in the sense of the present disclosure are aqueous solutions containing about 2 to about 70% by weight of a C₁-C₄ alcohol, more particularly ethanol or isopropanol. The agents as contemplated herein can additionally contain other organic solvents, such as methoxybutanol, benzyl alcohol, ethyl diglycol or 1,2-propylene glycol. Preferred are all water-soluble organic solvents.

Furthermore, a method for dyeing keratinous material, in particular human hair, comprising the following steps in the order given is particularly preferred:
(1-b) use of an agent on the keratinous material, wherein the agent contains at least one organic silicon compound from the group of silanes with one, two or three silicon atoms,
(2-b) washing off the agent
(3-a) use of an agent on the keratinous material, in which the agent contains at least one coated pigment with a colored core and a silicon-containing coating,
(4-a) washing off the agent.

Within the framework of this embodiment, the agent (b) applied on the keratin material in step (1) is first washed off in the next step (2), before the agent (a) is applied on the keratin material in the subsequent step (3). In step (4), the agent is then washed off.

Furthermore, a method for dyeing keratinous material, in particular human hair, comprising the following steps in the order given is particularly preferred:
(1-b) use of an agent on the keratinous material, wherein the agent contains at least one organic silicon compound from the group of silanes with one, two or three silicon atoms,
(2-b) no washing off the agent
(3-a) use of an agent on the keratinous material, in which the agent contains at least one coated pigment with a colored core and a silicon-containing coating,
(4-a) washing off the agent.

Within the framework of this embodiment, the agent (b) applied on the keratin material in step (1) is not washed off (step (2), but on the contrary in step (3) the agent (a) is applied on the keratin material with the agent (b) still present on it. Washing off then follows in step (4).

### Film-Forming Polymers

Furthermore, the agents used in step (a) and/or in step (b) of the method as invented additionally also contain at least one film-forming polymer.

The film-forming polymer(s) additionally contained are in this case not present in a pigment, as absorbed but are dissolved or else dispersed in the cosmetic carrier.

Polymers are macromolecules with a molecular weight of at least 1000 g/mol, preferably of at least 2500 g/mol, particularly preferably of at least 5000 g/mol, which include identical, repeating organic units. The polymers of the present disclosure may be synthetically produced polymers which are manufactured by polymerization of one type of monomer or by polymerization of different types of monomer which are structurally different from each other. If the polymer is produced by polymerizing a type of monomer, it is called a homo-polymer. If structurally different monomer types are used in polymerization, the resulting polymer is called a copolymer.

The maximum molecular weight of the polymer depends on the degree of polymerization (number of polymerized monomers) and the batch size and is determined by the polymerization method. For the purposes of the present disclosure, it is preferred that the maximum molecular weight of the film-forming hydrophobic polymer (c) is not more than 107 g/mol, preferably not more than 106 g/mol and particularly preferably not more than 105 g/mol.

As contemplated herein, a film-forming polymer is a polymer which is capable of forming a film on a substrate, for example on a keratinic material or a keratinic fibers. The formation of a film can be demonstrated, for example, by looking at the keratin material treated with the polymer under a microscope.

In a further preferred embodiment, a method as invented is exemplified in that the agent used in step (a) and/or in step (b) contains at least one film-forming polymer.

The agents used in step (a) and/or step (b) of the method as invented can be hydrophilic or hydrophobic film-forming polymers.

With the framework of a first embodiment, it can be preferable to use at least one hydrophobic, film-forming polymer.

A hydrophobic polymer is a polymer that has a solubility in water at 25° C. (760 mmHg) of less than 1% by weight.

The water solubility of the film-forming, hydrophobic polymer can be determined in the following way, for example. 1.0 g of the polymer is placed in a beaker. Make up to 100 g with water. A stir-fish is added, and the mixture is heated to 25° C. on a magnetic stirrer while stirring. It is stirred for 60 minutes. The aqueous mixture is then visually assessed. If the polymer-water mixture cannot be assessed visually due to a high turbidity of the mixture, the mixture is filtered. If a proportion of undissolved polymer remains on the filter paper, the solubility of the polymer is less than 1% by weight.

These include acrylic acid-type polymers, polyurethanes, polyesters, polyamides, polyureas, cellulose polymers, nitrocellulose polymers, silicone polymers, acrylamide-type polymers and polyisoprenes.

Particularly well suited film-forming, hydrophobic polymers are, for example, polymers from the group of copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

In a further preferred embodiment, an agent as contemplated herein is characterized in that it contains at least one film-forming hydrophobic polymer (c) selected from the group including copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

The film-forming hydrophobic polymers, which are selected from the group of synthetic polymers, polymers obtainable by radical polymerization or natural polymers, have proved to be particularly suitable for solving the problem as contemplated herein.

Other particularly well-suited film-forming hydrophobic polymers can be selected from the homopolymers or copolymers of olefins, such as cycloolefins, butadiene, isoprene or styrene, vinyl ethers, vinylamides, the esters or amides of (meth)acrylic acid with at least one C₁-C₂₀ alkyl group, an aryl group or a C2-C10 hydroxyalkyl group.

Other film-forming hydrophobic polymers may be selected from the homo- or copolymers of isooctyl (meth)acrylate; isononyl (meth)acrylate; 2-ethylhexyl (meth)acrylate; lauryl (meth)acrylate; isopentyl (meth)acrylate; n-butyl (meth)acrylate); isobutyl (meth)acrylate; ethyl (meth)acrylate; methyl (meth)acrylate; tert-butyl (meth)acrylate; stearyl (meth)acrylate; hydroxyethyl (meth)acrylate; 2-hydroxypropyl (meth)acrylate; 3-hydroxypropyl (meth)acrylate and/or mixtures thereof.

Other film-forming hydrophobic polymers may be selected from the homo- or copolymers of (meth)acrylamide; N-alkyl-(meth)acrylamides, in particular those with C2-C18 alkyl groups, such as N-ethyl-acrylamide, N-tert-butyl-acrylamide, le N-octyl-acrylamide; N-di(C1-C4)alkyl-(meth)acrylamide.

Other preferred anionic copolymers are, for example, copolymers of acrylic acid, methacrylic acid or their C₁-C₆ alkyl esters, as they are marketed under the INCI Declaration Acrylates Copolymers. A suitable commercial product is for example Aculyn0 33 from Rohm & Haas. Copolymers of acrylic acid, methacrylic acid or their C₁-C6 alkyl esters and the esters of an ethylenically unsaturated acid and an alkoxylated fatty alcohol are also preferred. Suitable ethylenically unsaturated acids are especially acrylic acid, methacrylic acid and itaconic acid; suitable alkoxylated fatty alcohols are especially steareth-20 or ceteth-20.

Some of the most preferred polymers on the market are Aculyn® 22 (acrylates/steareth-20 methacrylate copolymer), Aculyn® 28 (acrylates/Beheneth-25 methacrylate copolymer), Structure 2001® (acrylates/Steareth-20 Itaconate copolymer), Structure 3001® (acrylates/Ceteth-20 Itaconate copolymer), Structure Plus® (Acrylates/aminoacrylates C10-30 alkyl PEG-20 Itaconate copolymer), Carbopol® 1342, 1382, Ultrez 20, Ultrez 21 (acrylates/C10-30 alkyl acrylate crosspolymer), Synthalen W 2000® (acrylates/Palmeth-25 acrylate copolymer) or Soltex OPT (acrylates/C12-22 alkyl methacrylate copolymer) distribute by Rohme and Haas.

The homo- and copolymers of N-vinylpyrrolidone, vinylcaprolactam, vinyl-(C1-C6)alkyl-pyrrole, vinyl-oxazole, vinyl-thiazole, vinylpyrimidine, vinylimidazole can be named as suitable polymers based on vinyl monomers.

Furthermore, the copolymers octylacrylamide/acrylates/butylaminoethyl-methacrylate copolymer, as commercially marketed under the trade names AMPHOMER® or LOVOCRYL® 47 by NATIONAL STARCH, or the copolymers of acrylates/octylacrylamides marketed under the trade names DERMACRYL® LT and DERMACRYL® 79 by NATIONAL STARCH are particularly suitable.

Suitable olefin-based polymers include homopolymers and copolymers of ethylene, propylene, butene, isoprene and butadiene.

In another version, block copolymers can be used as film-forming hydrophobic polymers, which comprise at least one block of styrene or the derivatives of styrene. These block copolymers can be copolymers that contain one or more other blocks in addition to a styrene block, such as styrene/ethylene, styrene/ethylene/butylene, styrene/butylene, styrene/isoprene, styrene/butadiene. Such polymers are commercially distributed by BASF under the trade name “Luvitol HSB”.

Surprisingly, it was observed that especially intensive and washable dyes could be obtained, if the agent (b) contains at least one film-forming polymer selected from the group of copolymers of acrylic acid, homopolymers and copolymers of methacrylic acid, homopolymers and copolymers of acrylic acid esters, homopolymers or copolymers of methacrylic acid esters, the homopolymers or copolymers of acrylic acid amides, the homopolymers or copolymers of methacrylic acid amides, the copolymers of vinylpyrrolidones, the copolymers of vinyl alcohol, the copolymers of vinyl acetate, the homopolymers or copolymers of ethylene, the homopolymers or copolymers of propylene, the homopolymers or copolymers of styrene, polyurethanes, polyesters and/or polyamides.

In a further preferred embodiment, a method is characterized in that the film-forming polymer is selected from the group of homopolymers and copolymers of acrylic acid, homopolymers and copolymers of methacrylic acid, homopolymers and copolymers of acrylic acid esters, homopolymers and copolymers of methacrylic acid esters, homopolymers and copolymers of acrylic acid amides, homopolymers and copolymers of methacrylic acid amides, homopolymers and copolymers of vinylpyrrolidone, homopolymers and copolymers of vinyl alcohol, homopolymers and copolymers of vinyl acetate, homopolymers and copolymers of ethylene, homopolymers and copolymers of propylene, homopolymers and copolymers of styrene, polyurethanes, polyesters and/or polyamides.

Within the framework of a further embodiment, it can be preferred to insert at least one hydrophilic film-forming polymer in the agent used in the method as invented in step (a) and/or step (b).

A hydrophilic polymer is a polymer that has a solubility in water at 25° C. (760 mmHg) of more than 1% by weight, preferably more than 2% by weight.

The water solubility of the film-forming, hydrophilic polymer can be determined in the following way, for example. 1.0 g of the polymer is placed in a beaker. Make up to 100 g with water. A stir-fish is added and the mixture is heated to 25° C. on a magnetic stirrer while stirring. It is stirred for 60 minutes. The aqueous mixture is then visually assessed. A completely dissolved polymer appears macroscopically homogeneous. If the polymer-water mixture cannot be assessed visually due to a high turbidity of the mixture, the mixture is filtered. If no undissolved polymer remains on the filter paper, the solubility of the polymer is more than 1% by weight.

Nonionic, anionic and cationic polymers can be used as film-forming, hydrophilic polymers.

Suitable film-forming hydrophilic polymers can be selected, for example, from the group of polyvinylpyrrolidone (co)polymers, polyvinyl alcohol (co)polymers, vinyl acetate (co)polymers, carboxyvinyl (co)polymers, acrylic acid (co)polymers, methacrylic acid (co)polymers, natural gums, polysaccharides and/or acrylamide (co)polymers.

Furthermore, it is particularly preferred to use polyvinylpyrrolidone (PVP) and/or a vinylpyrrolidone-containing copolymer as film-forming hydrophilic polymer.

In another particularly preferred embodiment, an agent is characterized in that it contains (c) at least one film-forming, hydrophilic polymer selected from the group including polyvinylpyrrolidone (PVP) and the copolymers of polyvinylpyrrolidone.

It is further preferred if the agent as invented contains polyvinylpyrrolidone (PVP) as a film-forming, hydrophilic polymer. Surprisingly, the washing fastness of the coloration obtained with formulations containing PVP was also very good.

Particularly well suited polyvinyl pyrrolidones are, for example, available under the name Luviskol® K from BASF SE, especially Luviskol® K 90 or Luviskol® K 85 from BASF SE.

The polymer PVP K30, which is marketed by Ashland (ISP, POI Chemical), can also be used as another explicitly very well suited polyvinylpyrrolidone (PVP). PVP K 30 is a polyvinylpyrrolidone which is highly soluble in cold water and has the CAS number 9003-39-8. The molecular weight of PVP K 30 is about 40000 g/mol.

Other particularly suitable polyvinylpyrrolidones are the substances known under the trade names LUVITEC K 17, LUVITEC K 30, LUVITEC K 60, LUVITEC K 80, LUVITEC K 85, LUVITEC K 90 and LUVITEC K 115 and available from BASF.

The use of film-forming hydrophilic polymers from the group of copolymers of polyvinylpyrrolidone has also led to particularly good and washable color results.

Vinylpyrrolidone-vinyl ester copolymers, such as those marketed under the trademark Luviskol® (BASF), are particularly suitable film-forming hydrophilic polymers. Luviskol® VA 64 and Luviskol® VA 73, both vinylpyrrolidone/vinyl acetate copolymers, are particularly preferred non-ionic polymers.

Of the vinylpyrrolidone-containing copolymers, a styrene/VP copolymer and/or a vinylpyrrolidone-vinyl acetate copolymer and/or a VP/DMAPA acrylates copolymer and/or a VP/vinyl caprolactam/DMAPA acrylates copolymer are particularly preferred in cosmetic compositions.

Vinylpyrrolidone-vinyl acetate copolymers are marketed under the name Luviskol® VA by BASF SE. For example, a VP/Vinyl Caprolactam/DMAPA Acrylates copolymer is sold under the trade name Aquaflex® SF-40 by Ashland Inc. For example, a VP/DMAPA acrylates copolymer is marketed by Ashland under the name Styleze CC-10 and is a highly preferred vinylpyrrolidone-containing copolymer.

Other suitable copolymers of polyvinylpyrrolidone may also be the polymers obtained by conversion of N-vinylpyrrolidone with at least one further monomer from the group including V-vinyl formamide, vinyl acetate, ethylene, propylene, acrylamide, vinylcaprolactam, vinylcaprolactone and/or vinyl alcohol.

In an further quite especially embodiment, an agent as invented is characterized in that it contains at least one film-forming, hydrophilic polymer, which is selected from the group of polyvinylpyrrolidone (PVP), vinylpyrrolidone/vinylacetate copolymers, vinylpyrrolidone/styrene copolymers, vinylpyrrolidone/ethylene copolymers, vinylpyrrolidone/propylene copolymers, vinylpyrrolidone/vinylcaprolactam copolymers, vinylpyrrolidone/vinyl formamide copolymers and/or vinylpyrrolidone/vinyl alcohol copolymers.

Another fussy copolymer of vinylpyrrolidone is the polymer known under the INCI designation maltodextrin/VP copolymer.

Furthermore, intensively dyed keratin material, especially hair, with very good wash fastness could be obtained if a non-ionic, film-forming, hydrophilic polymer was used as the film-forming, hydrophilic polymer.

Within the framework of a further embodiment, the agents used in the method as invented contain at least one nonionic, film-forming, hydrophilic polymer.

As contemplated herein, a non-ionic polymer is understood to be a polymer which in a protic solvent—such as water—under standard conditions does not carry structural units with permanent cationic or anionic groups, which must be compensated by counter ions while maintaining electron neutrality. Cationic groups include quaternized ammonium groups but not protonated amines Anionic groups include carboxylic and sulfonic acid groups.

Particular preference is given to products containing, as a non-ionic, film-forming, hydrophilic polymer, at least one polymer selected from the group including
Polyvinylpyrrolidone,
Copolymers of N-vinylpyrrolidone and vinyl esters of carboxylic acids having 2 to 18 carbon atoms, in particular of N-vinylpyrrolidone and vinyl acetate,
Copolymers of N-vinylpyrrolidone and N-vinylimidazole and methacrylamide,
Copolymers of N-vinylpyrrolidone and N-vinylimidazole and acrylamide,
Copolymers of N-vinylpyrrolidone with N,N-di(C1 to C4)-alkylamino-(C2 to C4)-alkylacrylamide,

If copolymers of N-vinylpyrrolidone and vinyl acetate are used, it is again preferable if the molar ratio of the structural units contained in the monomer N-vinylpyrrolidone to the structural units of the polymer contained in the monomer vinyl acetate is in the range from about 20:80 to about 80:20, in particular from about 30:70 to about 60:40. Suitable copolymers of vinyl pyrrolidone and vinyl acetate are available, for example, under the trademarks Luviskol® VA 37, Luviskol® VA 55, Luviskol® VA 64 and Luviskol® VA 73 from BASF SE.

Another particularly preferred polymer is selected from the INCI designation VP/Methacrylamide/Vinyl Imidazole Copolymer, which is available under the trade name Luviset Clear from BASF SE.

Another particularly preferred non-ionic, film-forming, hydrophilic polymer is a copolymer of N-vinylpyrrolidone and N,N-dimethyl aminiopropyl methacrylamide, which is sold under the INCI designation VP/DMAPA Acrylates Copolymer e.g., under the trade name Styleze® CC 10 by ISP.

A cationic polymer of interest is the copolymer of N-vinylpyrrolidone, N-vinylcaprolactam, N-(3-dimethylaminopropyl)methacrylamide and 3-(methacryloylamino)propyl-lauryl-dimethylammonium chloride (INCI designation): Polyquaternium-69), which is marketed, for example, under the trade name AquaStyle® 300 (28-32 wt. % active substance in ethanol-water mixture, molecular weight 350000) by ISP.

Other suitable film-forming, hydrophilic polymers include
Vinylpyrrolidone-vinylimidazolium methochloride copolymers, as offered under the designations Luviquat® FC 370, FC 550 and the INCI designation Polyquaternium-16 as well as FC 905 and HM 552,
Vinylpyrrolidone-vinylcaprolactam-acrylate terpolymers, as they are commercially available with acrylic acid esters and acrylic acid amides as a third monomer component, for example under the name Aquaflex® SF 40.

Polyquaternium-11 is the reaction product of diethyl sulfate with a copolymer of vinyl pyrrolidone and dimethylaminoethyl methacrylate. Suitable commercial products are available under the names Dehyquart® CC 11 and Luviquat® PQ 11 PN from BASF SE or Gafquat 440, Gafquat 734, Gafquat 755 or Gafquat 755N from Ashland Inc.

Polyquaternium-46 is the reaction product of vinylcaprolactam and vinylpyrrolidone with methylvinyl imidazolium methosulfate and is available for example under the name Luviquat® Hold from BASF SE. Polyquaternium-46 is preferably used in an amount of 1 to 5% by weight—based on the total weight of the cosmetic composition. It particularly prefers to use polyquaternium-46 in combination with a cationic guar compound. It is even highly preferred that polyquaternium-46 is used in combination with a cationic guar compound and polyquaternium-11.

Suitable anionic film-forming, hydrophilic polymers can be, for example, acrylic acid polymers, which can be in non-cross-linked or cross-linked form. Such products are sold commercially under the trade names Carbopol 980, 981, 954, 2984 and 5984 by Lubrizol or under the names Synthalen M and Synthalen K by 3V Sigma (The Sun Chemicals, Inter Harz).

Examples of suitable film-forming, hydrophilic polymers from the group of natural gums are xanthan gum, gellan gum, carob gum.

Examples of suitable film-forming hydrophilic polymers from the group of polysaccharides are hydroxyethyl cellulose, hydroxypropyl cellulose, ethyl cellulose and carboxymethyl cellulose.

Suitable film-forming, hydrophilic polymers from the group of acrylamides are, for example, polymers which are produced from monomers of (methy)acrylamido-C1-C4-alkyl sulfonic acid or the salts thereof. Corresponding polymers may be selected from the polymers of polyacrylamidomethanesulfonic acid, polyacrylamidoethanesulfonic acid, polyacrylamidopropanesulfonic acid, poly2-acrylamido-2-methylpropanesulfonic acid, poly-2-methylacrylamido-2-methylpropanesulfonic acid and/or poly-2-methylacrylamido-n-butanesulfonic acid.

Preferred polymers of the poly(meth)arylamido-C1-C4-alkyl sulfonic acids are cross-linked and at least 90% neutralized. These polymers can be cross-linked or non-cross-linked.

Cross-linked and totally or partially neutralized polymers of the poly-2-acrylamido-2-methylpropane sulfonic acid type are known under the INCI designation “Ammonium Polyacrylamido-2-methyl

propanesulfonates” or “Ammonium Polyacryldimethyltauramides”.

Another preferred polymer of this type is the cross-linked poly-2-acrylamido-2-methyl-propanesulphonic acid polymer marketed by Clamant under the trade name Hostacerin AMPS, which is partially neutralized with ammonia.

In a further explicitly quite specifically preferred embodiment, a method as invented is characterized in that the agent used in step (a) and/or in step (b) contains at least one anionic, film-forming, polymer.

In this context, the best results can be obtained if the agent used in the step (a) and/or (b) contained at least one film-forming polymer that comprises at least one structural unit of the formula (P-I) and at least one structural unit of the formula (P-II)

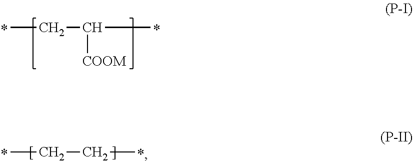

where
M stands for a hydrogen atom or for ammonium (NH₄), sodium, potassium, ½ magnesium or ½ calcium.

In a further preferred embodiment, a method as invented is exemplified that the film-forming polymer (at least one structural unit of the formula (P-I) and at least one structural unit of the formula (P-II)

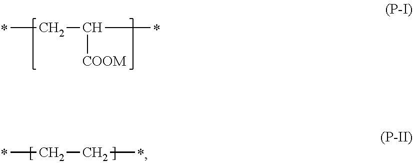

where
M stands for a hydrogen atom or for ammonium (NH₄), sodium, potassium, ½ magnesium or ½ calcium.

The film-forming hydrophobic polymer(s) as contemplated herein are preferably used in certain quantity ranges in the respective agent. In this context, it has proved to be particularly preferred for the solution of the task as contemplated herein if the agent (b)—based on the total weight of agent (b)—contains one or more polymers in a total amount of about 0.1 to about 18.0% by weight, preferably from about 1.0 to about 16.0% by weight, more preferably from about 5.0 to about 14.5% by weight and very particularly preferably from about 8.0 to about 12.0% by weight.

In a further preferred embodiment, a method as invented is exemplified in that the agent used in step (a) and/or (b) contains—respectively based on the total weight of the agent—one or more film-forming polymers in a total quantity of from about 0.1 to about 18.0% by weight, preferably from about 1.0 to about 16.0% by weight, more preferably from about 5.0 to about 14.5% by weight and quite particularly preferably from about 8.0 to about 12.0% by weight.

### pH Value and Alkalizing Agent

The agents used in step (a) and/or (b) of the method as invented are preferably set to a basic pH value. The pH value can be measured, for example, with a glass electrode, which is usually commercially available in the form of a combination electrode. Before measuring the pH value, the glass electrodes are usually calibrated with pH values known with calibration solutions. The pH values for the purposes of the present disclosure are pH values measured at a temperature of 22° C.

It is seen that it is possible to produce particularly resistant films on the keratin material on setting the alkali pH value to at least 9.6. Quite particularly good results could be obtained if the agent applied in the step (a) of the method were set to a pH value from about 9.7 to about 11.5%, preferably from about 9.8 to about 11.3, further preferably from about 9.9 to about 11.0 and specifically preferably from about 10.0 to about 10.9.

Furthermore, particularly good results could be obtained if the agent applied in the step (b) of the method were also set to a pH value from about 9.7 to about 11.5%, preferably from about 9.8 to about 11.3, further preferably from about 9.9 to about 11.0 and specifically preferably from about 10.0 to about 10.9.

The setting of the preferred alkaline pH value can be effected by using one or more alkalizing agents. Suitable alkalizing agents can be selected from the group including ammonia, alkanol amines, basic amino acids and inorganic alkalizing agents.

The alkanol amines which can be used in the agents can be selected, for example, from the group of primary amines having a C₂-C₆ alkyl base which carries at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol.

Similarly, good results could be obtained if the agent(s) applied in the method as alkalizing agents contained at least one basic amino acid.

For the purposes of the present disclosure, a basic amino acid is an organic compound which contains in its structure at least one protonatable amino group and at least one —COOH or —SO₃H group. Preferred amino acids are aminocarboxylic acids, especially α-(alpha)-aminocarboxylic acids and ω-aminocarboxylic acids, whereby α-aminocarboxylic acids are particularly preferred.

As contemplated herein, basic amino acids are those amino acids which have an isoelectric point pI of greater than 7.0.

Basic α-aminocarboxylic acids contain at least one asymmetric carbon atom. Within the framework of the present disclosure, both possible enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine and histidine, especially preferably arginine and lysine. In another particularly preferred embodiment, an agent as contemplated herein is therefore exemplified in that the alkalizing agent is a basic amino acid from the group arginine, lysine, ornithine and/or histidine.

Though the agents used in the method as invented are preferably set to pH values in the alkaline range, it can generally still be necessary to use acidifiers in small quantities to finely adjust the required pH value. Acidifiers suitable as contemplated herein are, for example, citric acid, lactic acid, acetic acid or even diluted mineral acids (such as hydrochloric acid, sulfuric acid, phosphoric acid).

### Other Ingredients

The agents (a) and (b) used in the previously described method may also contain one or more optional ingredients.

The products may also contain one or more surfactants. The term surfactants refers to surface-active substances. A distinction is made between anionic surfactants including a hydrophobic residue and a negatively charged hydrophilic head group, amphoteric surfactants, which carry both a negative and a compensating positive charge, cationic surfactants, which in addition to a hydrophobic residue have a positively charged hydrophilic group, and non-ionic surfactants, which have no charges but strong dipole moments and are strongly hydrated in aqueous solution.

Zwitterionic surfactants are those surface-active compounds which carry at least one quaternary ammonium group and at least one —COO⁽⁻⁾— or —SO₃⁽⁻⁾ group in the molecule. Particularly suitable zwitterionic surfactants are the so-called betaines such as the N-alkyl-N,N-dimethylammonium-glycinate, for example the cocoalkyl-dimethylammoniumglycinate, N-acylaminopropyl-N,N-dimethylammoniumglycinate, for example, cocoacylaminopropyl dimethyl ammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name cocamidopropyl betaine.

Ampholytic surfactants are surface-active compounds which, apart from a C8-C24 alkyl or acyl group, contain at least one free amino group and at least one —COOH— or —SO3H group in the molecule and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each with about 8 to 24 C atoms in the alkyl group. Typical examples of amphoteric or zwitterionic surfactants are alkylbetaines, alkylamidobetaines, amino-propionates, aminoglycinate, imidazoliniumbetaines and sulfobetaines.

Particularly preferred ampholytic surfactants are N-cocoalkylaminopropionate, cocoacylaminoethylaminopropionate and C12-C18 acylsarcosine.

The products may also additionally contain at least one non-ionic surfactant. Suitable non-ionic surfactants are alkyl polyglycosides as well as alkylene oxide addition products to fatty alcohols and fatty acids with 2 to 30 mol ethylene oxide per mol fatty alcohol or fatty acid. Preparations with good properties are also obtained if they contain as non-ionic surfactants fatty acid esters of ethoxylated glycerol reacted with at least 2 mol ethylene oxide. The non-ionic surfactants are used in a total quantity of about 0.1 to about 45% by weight, preferably from about 1 to about 30% by weight and very preferably from about 1 to about 15% by weight—based on the total weight of the respective agent.

In addition, the products may also contain at least one cationic surfactant. Cationic surfactants are surfactants, i.e., surface-active compounds, each with one or more positive charges. Cationic surfactants contain only positive charges. Usually, these surfactants are composed of a hydrophobic part and a hydrophilic head group, the hydrophobic part usually including a hydrocarbon backbone (e.g., including one or two linear or branched alkyl chains) and the positive charge(s) being located in the hydrophilic head group. Examples of cationic surfactants are
quaternary ammonium compounds which, as hydrophobic radicals, may carry one or two alkyl chains with a chain length of 8 to 28 C atoms,
quaternary phosphonium salts substituted with one or more alkyl chains with a chain length of 8 to 28 C atoms or
tertiary sulfonium salts.

Furthermore, the cationic charge can also be part of a heterocyclic ring (e.g., an imidazolium ring or a pyridinium ring) in the form of an onium structure. In addition to the functional unit carrying the cationic charge, the cationic surfactant may also contain other uncharged functional groups, as is the case for example with esterquats. The cationic surfactants are used in a total quantity of about 0.1 to about 45 wt. %, preferably from about 1 to about 30 wt. % and most preferably from about 1 to about 15 wt. %—based on the total weight of the respective agent.

Furthermore, the means as contemplated herein may also contain at least one anionic surfactant. Anionic surfactants are surface-active agents with exclusively anionic charges (neutralized by a corresponding counter cation). Examples of anionic surfactants are fatty acids, alkyl sulphates, alkyl ether sulphates and ether carboxylic acids with 12 to 20 C atoms in the alkyl group and up to 16 glycol ether groups in the molecule.

The anionic surfactants are used in a total quantity of about 0.1 to about 45 wt. %, preferably from about 1 to about 30 wt. % and most preferably from about 1 to about 15 wt. %—based on the total weight of the respective agent.

They may also contain other active substances, auxiliaries and additives, such as solvents, fatty components such as C₈-C₃₀-fatty alcohols, C₈-C₃₀-fatty acid triglycerides, C₈-C₃₀-fatty acid monoglycerides, C₈-C₃₀-fatty acid diglycerides and/or hydrocarbons, structural agents such as glucose, maleic acid and lactic acid; hair conditioning compounds such as phospholipids, for example lecithin and cephalins; perfume oils, dimethylisosorbide and cyclodextrins; fiber structure-improving active substances, in particular mono-, di- and oligosaccharides such as glucose, galactose, fructose, fructose and lactose; dyes for coloring the composition; anti-dandruff active substances such as Piroctone Olamine, Zinc Omadine and Climbazol; amino acids and oligopeptides; protein hydrolysates on animal and/or vegetable basis, as well as in the form of their fatty acid condensation products or optionally anionic or cationically modified derivatives; vegetable oils; sunscreens and UV-blockers; active ingredients such as panthenol, pantothenic acid, pantolactone, allantoin, pyrrolidinonecarboxylic acids and their salts, and bisabolol; polyphenols, in particular hydroxycinnamic acids, 6,7-dihydroxycumarine, hydroxybenzoic acids, catechine, tannine, leukoanthocyanidine, anthocyanidine, flavanone, flavone and flavonols; ceramides or pseudoceramides; vitamins, provitamins and vitamin precursors; plant extracts; fats and waxes such as fatty alcohols, beeswax, montan wax and paraffins; swelling and penetrating substances such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescent agents such as ethylene glycol mono- and distearate and PEG-3 distearate; and blowing agents such as propane-butane mixtures, N₂O, dimethyl ether, CO₂ and air.

The selection of these other substances will be made by the specialist according to the desired properties of the agents. With regard to other optional components and the quantities of these components used, explicit reference is made to the relevant manuals known to the specialist. The additional active ingredients and auxiliary substances are preferably used in the preparations as contemplated herein in quantities of about 0.0001 to about 25 wt. % each, in particular from about 0.0005 to about 15 wt. %, based on the total weight of the respective agent.

### Agent for Dyeing Keratin Materials

Within the framework of a particularly preferred embodiment, the user can use one and the same agent for the steps (a) and (b), which is then exemplified in that it contains

(a) at least one coated pigment with a colored core and a silicon-containing coating. and

(b) at least one organic silicon compound from the group of silanes having one, two or three silicon atoms.

A second object of the present disclosure is therefore an agent for coloring keratinous material, particularly human hair, containing
(a) at least one coated pigment with a colored core and a silicon-containing coating. and
(b) at least one organic silicon compound from the group of silanes having one, two or three silicon atoms,
Wherein the coated pigment and the organic silicon compound from the group of silanes with one, two or three silicon atoms have already been revealed in the description of the first object of the present disclosure, in detail.

Multi-Component Packaging Unit (Kit-of-Parts)

Within the framework of a further preferred embodiment, the user can also use two different agents for the steps (a) and (b) of the method. In order to increase user comfort, the user is preferably provided with these agents in the form of a multi-component packaging unit (kit-of-parts).

A third object of the present disclosure is therefore a multi-component packaging unit (kit-of-parts) for treating keratinous material, comprehensively packaged separately from one another:
a first container with an agent (a), containing at least one coated pigment with a colored core and a silicon-containing coating. and
a second container with an agent (b) containing at least one organic silicon compound selected from the group including silanes having one, two or three silicon atoms,
Wherein the coated pigment and the organic silicon compound from the group of silanes with one, two or three silicon atoms have already been revealed in the description of the first object of the present disclosure, in detail.

The coated pigments contained in the agent (a) of the kit correspond to the coated pigments that were also used in step (a) of the previously described method.

The organic silicon compounds from the group of silanes with one, two or three silicon atoms contained in agent (b) of the kit correspond to the organic silicon compounds that were also used in step (b) of the previously described method.

With respect to the other preferred embodiments of the agent as invented and multi-component packaging unit as contemplated herein, what was stated regarding the method as invented applies mutatis mutandis.

### EXAMPLES

### Example 1: Coating of a Pigment

20.0 g poly(4-styrene sulfonate), sodium salt were dissolved in 500 ml water under stirring. Thereafter, with constant stirring, 10,0 g red color pigment (CI 12085, 1-[(2-Chloro-4-nitrophenyl)azo]-2-naphthol) is added. This mix was then ground in a ball mill for 60 minutes at room temperature (3500 revolutions per minute). This suspension was then centrifuged three times to separate the superfluous anionic polymer and washed with distilled water.

The solid obtained in this manner was redispersed in 500 ml water. Then, 10.0 g of Poly(dimethyl diallyl ammonium chloride) (Polyquaternium-6) was added. It was stirred for 20 minutes more at room temperature. This suspension is then again centrifuged three or to separate the superfluous cationic polymer and then washed with distilled water.

5.0 of the pigment coated in this manner were now dispersed in 35.0 g distilled water at room temperature. This mixture was then diluted with 200 ml of ethanol. Thereafter, 200 mg polyvinyl pyrrolidone and 0.15 g of ammonia were added. Thereafter, 5.0 g of tetraethoxy silane in 40 ml ethanol were now added to this mixture within 60 minutes in small portions. The mixture was thereafter stirred for 24 hours at room temperature. To separate the coated pigments, they are again centrifuged three to four times and washed with distilled water. The solid obtained in this manner was dried in vacuum at room temperature for a few days.

### Example 2

The following formulations were produced:(all details are in % by weight, unless otherwise stated).

| Agent (I) | in weight % |
|---|---|
| Coated red pigment CI 12085 from example 1 | 10.0 |
| Ethylene/sodium acrylate copolymer (b1) | 40.0 |
| 25% solution | |
| Water | ad 100 |

| Agent (II) | in weight % |
|---|---|
| (3-Aminopropyl)triethoxysilane | 20.0 |
| Methyltrimethoxysilane | 70.0 |
| Water | ad 100 |

The agents (I) and (I) are mixed with each other in the ratio 1:1 just before use. The pH value of the resultant medium was adjusted to a value of 10.5 by adding ammonia or lactic acid. The medium was left to stand for about 5 minutes.

This medium was then applied on the hair strands (Kerling, Euronatural hair white), briefly massaged in and allowed to act for about 1 minute. The medium was then rinsed off with water.

An intensive red coloration of the hair strands with good washability was obtained.

### Example 3

The following formulations were used:

| Agent (I) | in weight % |
|---|---|
| Coated red pigment CI 12085 from example 1 | 10.0 |
| Ethylene/sodium acrylate copolymer (b1) | 40.0 |
| 25% solution | |
| Water | ad 100 |

| Agent (II) | in weight % |
|---|---|
| (3-Aminopropyl)triethoxysilane | 20.0 |
| Methyltrimethoxysilane | 70.0 |
| Water | ad 100 |

The medium (II) was first applied on the hair strands (Kerling, Euronatural hair white), briefly massaged in and allowed to act for about 1 minute. The medium (II) was then rinsed off with water.

Thereafter, the medium (I) was applied on the hair strands, allowed to act for 1 minute and then similarly rinsed off water.

An intensive red coloration of the hair strands with good washability was obtained.

### Example 4

The following formulations were used:

| Agent (I) | in weight % |
|---|---|
| Coated red pigment CI 12085 from example 1 | 10.0 |
| Ethylene/sodium acrylate copolymer (b1) | 40.0 |
| 25% solution | |
| Water | ad 100 |

| Agent (II) | in weight % |
|---|---|
| (3-Aminopropyl)triethoxysilane | 20.0 |
| Methyltrimethoxysilane | 70.0 |
| Water | ad 100 |

The medium (I) was first applied on the hair strands (Kerling, Euronatural hair white), briefly massaged in and allowed to act for about 1 minute. The medium (I) was then rinsed off with water.

Thereafter, the medium (II) was applied on the hair strands, allowed to act for 1 minute and then similarly rinsed off water.

This also resulted in hair strands having an intensive, red coloration with good washability.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Method for dyeing keratinous material comprising the following steps:
(a) applying a first agent on the keratinous material, in which the first agent comprises at least one coated pigment with a colored core and a silicon-containing coating, and
(b) applying an aa second agent on the keratinous material, wherein the second agent comprises at least one organic silicon compound selected from the group of silanes with one, two or three silicon atoms.

2. (canceled)

3. Method as claimed in claim 1, wherein the coated pigment comprises a core of an organic material selected from the group consisting of carmine, quinacridone, phthalocyanine, sorghum, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, and red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 or CI 75470.

4. Method as claimed in claim 1, wherein the coating of the coated pigments is obtained by means of a surface treatment using a surface treatment agent, wherein the surface treatment agent is selected from the group of tetraalkoxy silanes, alkyltrialkoxy silanes, dialkyl dialkoxy silanes, and trialkylalkoxy silanes.

5. A method as claimed in claim 4, wherein the surface treatment agent is selected from the group of the tetraalkoxy silanes of the formula (O-I), 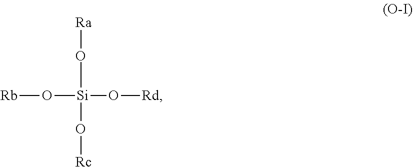
where
Ra, Rb, Rc and Rd mutually independently represent a linear or branched, saturated or unsaturated C₁-C₁₂-alkyl group.

6. Method as claimed in claim 1, wherein the first agent comprises at least one multiple coated pigment with a colored core and a silicon-containing outer coating.

7. Method according to claim 6, wherein the first agent comprises at least one multiple coated pigment that comprises the following layers:
(S1) optionally a coating with an anionic polymer,
(S2) optionally a coating with a cationic polymer,
(S3) optionally a coating with a nonionic polymer, and
(S4) a coating that is obtained by means of surface treatment with a surface treatment agent, in which the surface treatment agent is selected from the group of the tetra alkoxy silanes, alkyl trialkoxy silanes, dialkyl dialkoxy silanes, and trialkyl alkoxy silanes,
with the proviso, that the pigment possesses at least one of the coatings (S1), (S2) and (S3).

8. Method according to claim 6, wherein the first agent comprises at least one multiple coated pigment that comprises the following layers:
(S1) a first coating with an anionic polymer,
(S2) a second coating with a cationic polymer,
(S3) a third coating with a nonionic polymer and
(S4) a fourth coating that is obtained by means of a surface treatment with a surface treatment agent, where the surface treatment agent is selected from the group of tetra alkoxy silanes, of alkyl trialkoxy silanes, of the dialkyl dialkoxy silanes and of trialkyl alkoxy silanes.

9. Method as claimed in claim 7, wherein the coated pigment comprises:
(S1) a coating with an anionic polymer selected from the group consisting of homo and copolymers of styrene-4-sulfonic acid, homo and copolymers of acrylic acid, homo and copolymers of methacrylic acid, homo and copolymers of crotonic acid, homo and copolymers of maleic acid, homo and copolymers of 2-acrylamido-2-methyl propane sulfonic acid, and their physiologically compatible salts.

10. Method as claimed in claim 7, characterized in thatwherein the coated pigment comprises:
(S2) a coating with a cationic polymer selected from the group consisting of homo and copolymers of dimethyl diallyl ammonium salts, homo- and copolymers of tri-C₁-C₆-alkyl-methacryloxy-C₁-C₆-alkyl-ammonium salts, homo- and copolymers of tri-C₁-C₆-alkyl-acryloxy-C₁-C₆-alkyl ammonium salts, and homo- and copolymers of 1-vinyl-3-(C₁-C₆-alkyl) imidazolium salts.

11. Method as claimed in claim 7, characterized in thatwherein the coated pigment comprises:
(S3) a coating with a nonionic polymer from the group of the homo and copolymers of vinyl pyrrolidones, homo and copolymers of vinyl acetate, homo and copolymers of styrene, homo and copolymers of ethene, and homo and copolymers of vinyl alcohol.

12. (canceled)

13. Method as claimed in claim 1, wherein the second agent comprises at least one organic silicon compound of the formula (I) and
R₁R₂N-L-Si(OR₃)ₐ(R₄)_{b} (I),
where
R₁, R₂ both represent a hydrogen atom, and L represents a linear, divalent C₁-C₆-alkylene group,
R3, R4 independently represent a methyl group or an ethyl group, and
a stands for the number 1 to 3 and
b stands for the number 3−a.

14. Method as claimed in claim 13, wherein the second agent comprises at least one organic silicon compound selected from the group consisting of
(3-Aminopropyl)trimethoxysilane
(3-Aminopropyl)triethoxysilane
(2-Aminoethyl)trimethoxysilane
(2-Aminoethyl)triethoxysilane
(3-Dimethylaminopropyl)trimethoxysilane
(3-Dimethylaminopropyl)triethoxysilane
(2-Dimethylaminoethyl)trimethoxysilane, and
(2-Dimethylaminoethyl)triethoxysilane.

15. Method as claimed in claim 1, wherein the second agent comprises at least one organic silicon compound of the formula (II)
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
where
e and f both stand for the number 1,
g and h both stand for the number 0,
A and A′ independently represent a linear, divalent C₁-C₆ alkylene group and
R7 represents a hydrogen atom, a methyl group, a 2-hydroxyethyl group, a 2-alkenyl group, a 2-aminoethyl group, or a group of formula (III),
-(A″″)-Si(R6″)_{d}″(OR₅″)_{c″} (III),
c, stands for an integer from about 1 to about 3,
d stands for the integer 3−c,
c′ stands for an integer from about 1 to about 3,
d′ stands for the integer 3−c′,
c″ stands for an integer from about 1 to about 3,
d″ stands for the integer 3−c″,
e stands for 0 or 1,
f stands for 0 or 1,
g stands for 0 or 1,
h stands for 0 or 1,
provided that at least one of e, f, g and h is different from 0.

16. Method as claimed in claim 15, wherein the second agent comprises at least one organic silicon compound selected from the group consisting of
3-(trimethoxysilyl)-N-[3-(trimethoxysilyl) propyl]-1-propane amine
3-(Triethoxysilyl)-N-[3-(triethoxysilyl) propyl]-1-propanamine
N-methyl-3-(trimethoxysilyl)-N-[3-(trimethoxysilyl) propyl]-1-propanamine
N-Methyl-3-(triethoxysilyl)-N-[3-(triethoxysilyl) propyl]-1-propanamine
2-[Bis[3-(trimethoxysilyl) propyl]amino]-ethanol
2-[bis[3-(triethoxysilyl) propyl]amino]ethanol
3-(Trimethoxysilyl)-N,N-bis[3-(trimethoxysilyl) propyl]-1-propanamine
3-(Triethoxysilyl)-N,N-bis[3-(triethoxysilyl) propyl]-1-propanamine
N1,N1-bis[3-(trimethoxysilyl) propyl]-1,2-ethanediamine,
N1,N1-bis[3-(triethoxysilyl) propyl]-1,2-ethanediamine,
N,N-bis[3-(trimethoxysilyl)propyl]-2-propene-1-amine, and
N,N-bis[3-(triethoxysilyl)propyl]-2-propene-1-amine.

17. Method as claimed in claim 13, wherein the second agent further comprises at least one organic silicon compound of the formula (IV)
R₉Si(OR₁₀)ₖ(R₁₁)ₘ (IV),
where
R₉ represents a C₁-C₁₂ alkyl group,
R₁₀ represents a hydrogen atom or a C₁-C₆ alkyl group,
R₁₁ represents a C₁-C₆ alkyl group
k is an integer from about 1 to about 3, and
m stands for the integer 3−k.

18. Method as claimed in claim 17, wherein the second agent comprises at least one organic silicon compound selected from the group consisting of
Methyltrimethoxysilane
Methyltriethoxysilane
Ethyltrimethoxysilane
Ethyltriethoxysilane
Octyltrimethoxysilane
Octyltriethoxysilane
Dodecyltrimethoxysilane, and
Dodecyltriethoxysilane.

19. (canceled)

20. Method as claimed in claim 1 comprising applying the agents used in step (a) and in step (b) simultaneously or successively on the keratinous material.

21. (canceled)

22. (canceled)

23. (canceled)

24. Agent for dyeing keratinous material, comprising
(a) at least one coated pigment with a colored core and a silicon-containing coating. and
(b) at least one organic silicon compound from the group of silanes having one, two or three silicon atoms.

25. Multi-component packaging unit (Kit-of-parts) for the treatment of keratinous material, comprising separately packaged:
a first container with an agent (a), comprising at least one coated pigment with a colored core and a silicon-containing coating, and
a second container with an agent (b) comprising at least one organic silicon compound selected from the group consisting of silanes having one, two or three silicon atoms.
